(19) United States
(12) Patent Application Publication
(10) Pub. No.: US 2013/0296791 A1
(43) Pub. Date: Nov. 7, 2013

(54) MEDICATION DELIVERY ASSEMBLY

(71) Applicants: Michael Segev, Mizpe Netofa (IL); Alex Zislin, Naharia (IL)

(72) Inventors: Michael Segev, Mizpe Netofa (IL); Alex Zislin, Naharia (IL)

(21) Appl. No.: 13/997,669

(22) PCT Filed: Jan. 18, 2012
(86) PCT No.: PCT/IB12/50237

§ 371 Date: Jul. 10, 2013

Related U.S. Application Data
(60) Provisional application No. 61/433,538, filed on Jan. 18, 2011.

(51) Int. Cl. A61M 37/00; A61M 5/32

(52) U.S. Cl. CPC A61M 37/0015; A61M 5/3202
USPC 604/173; 604/192; 604/272

(57) ABSTRACT

A drug delivery assembly for use in association with an outlet port of an injection device, the assembly including a connector for association with the outlet port of the injection device, a skin interface element including a fluid flow channel in fluid connection with at least one hollow penetrating element deployed for penetrating into a biological barrier and a shield deployed to prevent inadvertent contact with said hollow penetrating element prior to use. The shield being retained in engagement with at least one of the connector and the skin interface element. The skin interface element is mechanically engaged with the connector so as to be displaceable relative to the connector between an inactive position and an active position. The motion of the skin interface element relative to the connector from the inactive position to the active position is effective to disengage retention of the shield.

### REFERENCE TO RELATED APPLICATIONS

Reference is made to PCT Patent Application WO2010067319A3, filed Dec. 9, 2009 and entitled “DEVICE FOR INJECTING FLUID ISOLATED FROM MICRONEEDLE HUB WITH DEAD-SPACE-REDUCING INSERT”, the disclosures of which is hereby incorporated by reference.

Reference is further made to U.S. Provisional Patent Application Ser. No. 61/433,538, filed Jan. 18, 2011 and entitled “Needle Safety Device”, the disclosure of which is hereby incorporated by reference and priority of which is hereby claimed pursuant to 37 CFR 1.78(a) (4) and (5)(i).

### FIELD OF THE INVENTION

The present invention relates to medication delivery assemblies and more particularly to medication delivery assemblies for injection devices.

### BACKGROUND OF THE INVENTION

The following publications are believed to represent the current state of the art: U.S. Pat. Nos. 5,232,454; 5,447,501; 5,665,075; 6,406,459; 6,632,199; 6,719,732; 7,241,277; 7,300,421; 7,387,617; 7,530,965; 7,537,581; 7,798,994;

U.S. Patent Publication Nos. 20070016141; 20090012478; 20090105661; 20090062744; 20100137810; 20100222749; 4202334; 20020045864; 5785691; 20100274185A1.

### SUMMARY OF THE INVENTION

The present invention seeks to provide an improved medication delivery assembly. There is thus provided in accordance with a preferred embodiment of the present invention a drug delivery assembly for use in association with an outlet port of an injection device, the assembly including a connector for association with the outlet port of the injection device, a skin interface element including a fluid flow channel in fluid connection with at least one hollow penetrating element deployed for penetrating into a biological barrier, and a shield deployed to prevent inadvertent contact with the hollow penetrating element prior to use, the shield being retained in engagement with at least one of the connector and the skin interface element.

The skin interface element is mechanically engaged with the connector so as to be displaceable relative to the connector between an inactive position in which the fluid flow channel is isolated from the outlet port of the injection device and an active position in which the fluid flow channel is in fluid connection with the outlet port of the injection device. The motion of the skin interface element relative to the connector from the inactive position to the active position is effective to disengage retention of the shield.

### BRIEF DESCRIPTION OF THE DRAWINGS

The present invention will be understood and appreciated more fully from the following detailed description, taken in conjunction with the drawings in which:

FIG. 1 is a simplified pictorial illustration of a medication delivery assembly constructed and operative in accordance with a preferred embodiment of the invention;

FIGS. 2A and 2B are simplified respective detailed front and rear sub assembly exploded view illustrations of the medication delivery assembly of FIG. 1;

FIG. 3 is a simplified pictorial view of a connector element forming part of the medication delivery assembly of FIG. 1;

FIGS. 3A and 3B are simplified respective side view and sectional illustrations of the connector element of the medication delivery assembly of FIG. 1, FIG. 3B being taken along lines D-D in FIG. 3A;

FIGS. 3C and 3D are simplified respective side view and sectional illustrations of the connector element of the medication delivery assembly of FIG. 1,

FIG. 3D being taken along lines B-B in FIG. 3C, perpendicular to lines D-D in FIG. 3A;

FIG. 3E is a simplified bottom view of the connector element of the medication delivery assembly of FIG. 1;

FIG. 4 is a simplified pictorial view of a septum element forming part of the medication delivery assembly of FIG. 1;

FIGS. 4A and 4B are simplified respective side view and sectional illustrations of the septum element of the medication delivery assembly of FIG. 1, FIG. 4B being taken along lines A-A in FIG. 4A;

FIG. 5 is a simplified pictorial view of a skin interface element forming part of the medication delivery assembly of FIG. 1;

FIGS. 5A and 5B are simplified respective side view and sectional illustrations of the skin interface element of the medication delivery assembly of FIG. 1, FIG. 5B being taken along lines C-C in FIG. 5A;

FIGS. 5C and 5D are simplified respective side view and sectional illustrations of the skin interface element of the medication delivery assembly of FIG. 1, FIG. 5D being taken along lines E-E in FIG. 5C, perpendicular to lines C-C in FIG. 5A;

FIG. 5E is a simplified bottom view of the skin interface element forming part of the medication delivery assembly of FIG. 1;

FIG. 6 is a simplified pictorial view of a shield element forming part of the medication delivery assembly of FIG. 1;

FIGS. 6A and 6B are simplified respective side view and sectional illustrations of the shield element of the medication delivery assembly of FIG. 1, FIG. 6B being taken along lines F-F in FIG. 6A;

FIGS. 6C and 6D are simplified respective side view and sectional illustrations of the shield element of the medication delivery assembly of FIG. 1,

FIG. 6D being taken along lines B-B in FIG. 6C, perpendicular to lines F-F in FIG. 6A;

FIGS. 7A and 7B are simplified respective side view and sectional illustrations of the medication delivery assembly of FIG. 1 in an inactive operative position. FIG. 7B being taken along lines I-I in FIG. 7A;

FIG. 7C is a simplified partial enlargement of FIG. 7B;

FIGS. 7D and 7E are simplified respective side view and sectional illustrations of the medication delivery assembly of FIG. 1 in an inactive operative position. FIG. 7E being taken along lines K-K in FIG. 7D, perpendicular to lines I-I in FIG. 7A;

FIG. 7F is a simplified partial enlargement of FIG. 7E;

FIG. 8A is a simplified partial enlargement of sectional illustration of the medication delivery assembly of FIG. 1 in an active operative position, taken along lines I-I in FIG. 7A;

FIG. 8B is a simplified partial enlargement of sectional illustration of the medication delivery assembly of FIG. 1 in an active operative position, taken along lines K-K in FIG. 7D, perpendicular to lines I-I in FIG. 7A;

FIG. 9A is a simplified partial enlargement of sectional illustration of the medication delivery assembly of FIG. 1 in an open operative position, taken along lines I-1 in FIG. 7A;

FIG. 9B is a simplified partial enlargement of sectional illustration of the medication delivery assembly of FIG. 1 in an open operative position, taken along lines K-K in FIG. 7D, perpendicular to lines I-I in FIG. 7A;

FIG. 10A is a simplified partial enlargement of sectional illustration of the medication delivery assembly of FIG. 1 in an injection operative position, taken along lines I-I in FIG. 7A;

FIG. 10B is a simplified partial enlargement of sectional illustration of the medication delivery assembly of FIG. 1 in an injection operative position, taken along lines K-K in FIG. 7D, perpendicular to lines I-I in FIG. 7A;

FIG. 11 is a simplified pictorial illustration of a medication delivery assembly constructed and operative in accordance with another preferred embodiment of the invention;

FIGS. 12A and 12B are simplified respective detailed front and rear sub assembly exploded view illustrations of the medication delivery assembly of FIG. 11;

FIG. 13 is a simplified pictorial view of a connector element forming part of the medication delivery assembly of FIG. 11;

FIGS. 13A and 13B are simplified respective side view and sectional illustrations of the connector element of the medication delivery assembly of FIG. 11, FIG. 13B being taken along lines A-A in FIG. 13A;

FIGS. 13C and 13D are simplified respective side view and sectional illustrations of the connector element of the medication delivery assembly of FIG. 11,

FIG. 13D being taken along lines C-C in FIG. 13C, perpendicular to lines A-A in FIG. 13A;

FIG. 13E is a simplified bottom view of the connector element of the medication delivery assembly of FIG. 11;

FIG. 14 is a simplified pictorial view of a septum element forming part of the medication delivery assembly of FIG. 11;

FIGS. 14A and 14B are simplified respective side view and sectional illustrations of the septum element of the medication delivery assembly of FIG. 11, FIG. 14B being taken along lines D -Din FIG. 14A;

FIG. 15 is a simplified pictorial view of a skin interface element forming part of the medication delivery assembly of FIG. 11;

FIGS. 15A and 15B are simplified respective side view and sectional illustrations of the skin interface element of the medication delivery assembly of FIG. 11, FIG. 15B being taken along lines B-B in FIG. 15A;

FIGS. 15C and 15D are simplified respective side view and sectional illustrations of the skin interface element of the medication delivery assembly of FIG. 11, FIG. 15D being taken along lines C-C in FIG. 15C, perpendicular to lines B-B in FIG. 15A;

FIG. 15E is a simplified bottom view of the skin interface element forming part of the medication delivery assembly of FIG. 11;

FIG. 16 is a simplified pictorial view of a shield element forming part of the medication delivery assembly of FIG. 11;

FIGS. 16A and 16B are simplified respective side view and sectional illustrations of the shield element of the medication delivery assembly of FIG. 11, FIG. 16B being taken along lines B-B in FIG. 16A;

FIGS. 16C and 16D are simplified respective side view and sectional illustrations of the shield element of the medication delivery assembly of FIG. 11,

FIG. 16D being taken along lines D-D in FIG. 16C, perpendicular to lines B-B in FIG. 16A;

FIGS. 17A and 17B are simplified respective side view and sectional illustrations of the medication delivery assembly of FIG. 11 in an inactive position. FIG. 17B being taken along lines A-A in FIG. 17A;

FIG. 17C is a simplified partial enlargement of FIG. 17B;

FIGS. 17D and 17E are simplified respective side view and sectional illustrations of the medication delivery assembly of FIG. 11 in an inactive operative position. FIG. 17E being taken along lines E-E in FIG. 17D, perpendicular to lines A-A in FIG. 17A;

FIG. 17F is a simplified partial enlargement of FIG. 17E;

FIG. 18A is a simplified partial enlargement of sectional illustration of the medication delivery assembly of FIG. 11 in a first active operative position, taken along lines A-A in FIG. 17A;

FIG. 18B is a simplified partial enlargement of sectional illustration of the medication delivery assembly of FIG. 1 in a first active operative position, taken along lines E-E in FIG. 17D, perpendicular to lines A-A in FIG. 17A;

FIG. 19A is a simplified partial enlargement of sectional illustration of the medication delivery assembly of FIG. 1 in a second active operative position, taken along lines A-A in FIG. 17A;

FIG. 19B is a simplified partial enlargement of sectional illustration of the medication delivery assembly of FIG. 1 in a second active operative position, taken along lines E-E in FIG. 17D, perpendicular to lines A-A in FIG. 17A;

FIG. 20A is a simplified partial enlargement of sectional illustration of the medication delivery assembly of FIG. 11 in an open operative position, taken along lines A-A in FIG. 17A;

FIG. 20B is a simplified partial enlargement of sectional illustration of the medication delivery assembly of FIG. 11 in an open operative position, taken along lines E-E in FIG. 17D, perpendicular to lines A-A in FIG. 17A;

FIG. 21A is a simplified partial enlargement of sectional illustration of the medication delivery assembly of FIG. 11 in an injection operative position, taken along lines A-A in FIG. 17A;

FIG. 21B is a simplified partial enlargement of sectional illustration of the medication delivery assembly of FIG. 11 in an injection operative position, taken along lines E-E in FIG. 17D, perpendicular to lines A-A in FIG. 17A;

FIG. 22A and 22B are simplified enlargement orthogonal cross sectional view illustrations of a medication delivery assembly in an inactive operative position constructed and operative in accordance with another preferred embodiment of the invention;

FIG. 23A and 23B are simplified enlargement orthogonal cross sectional view illustrations of a medication delivery assembly of FIGS. 22A and 22B.

### DETAILED DESCRIPTION OF PREFERRED EMBODIMENTS

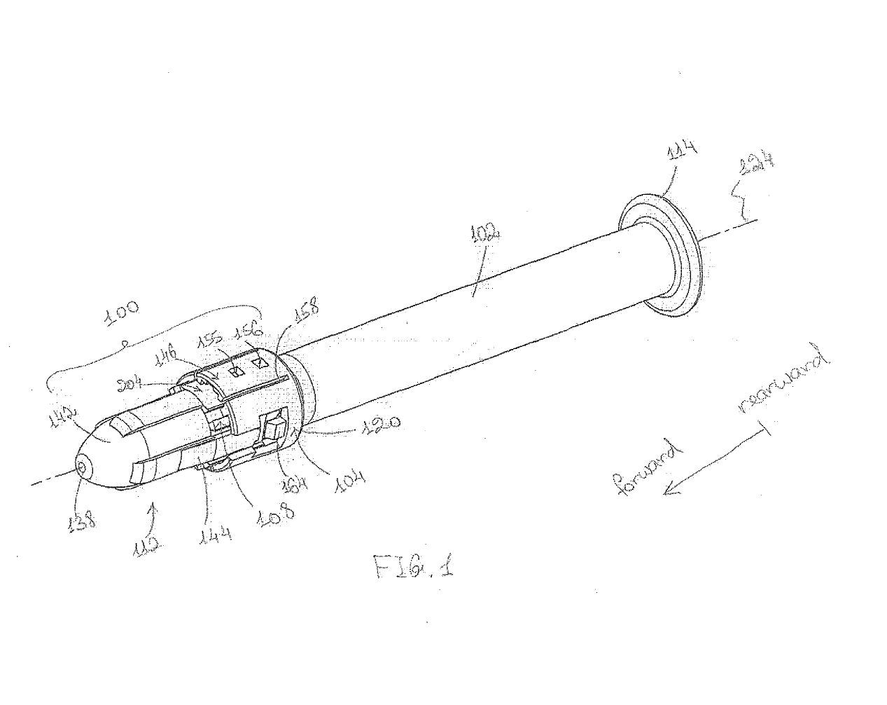

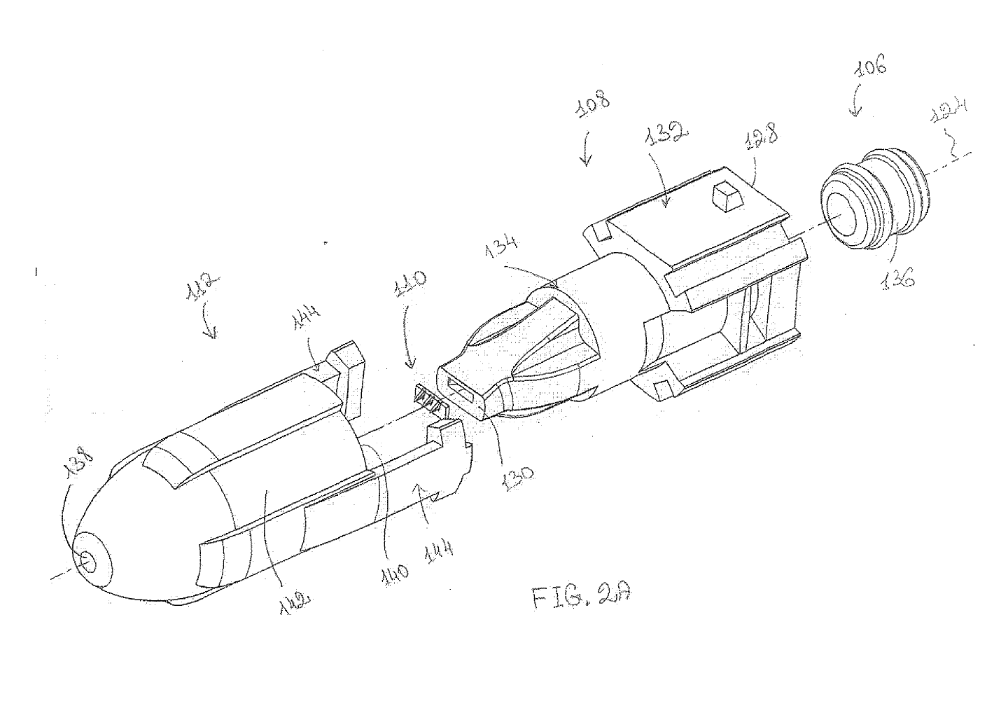

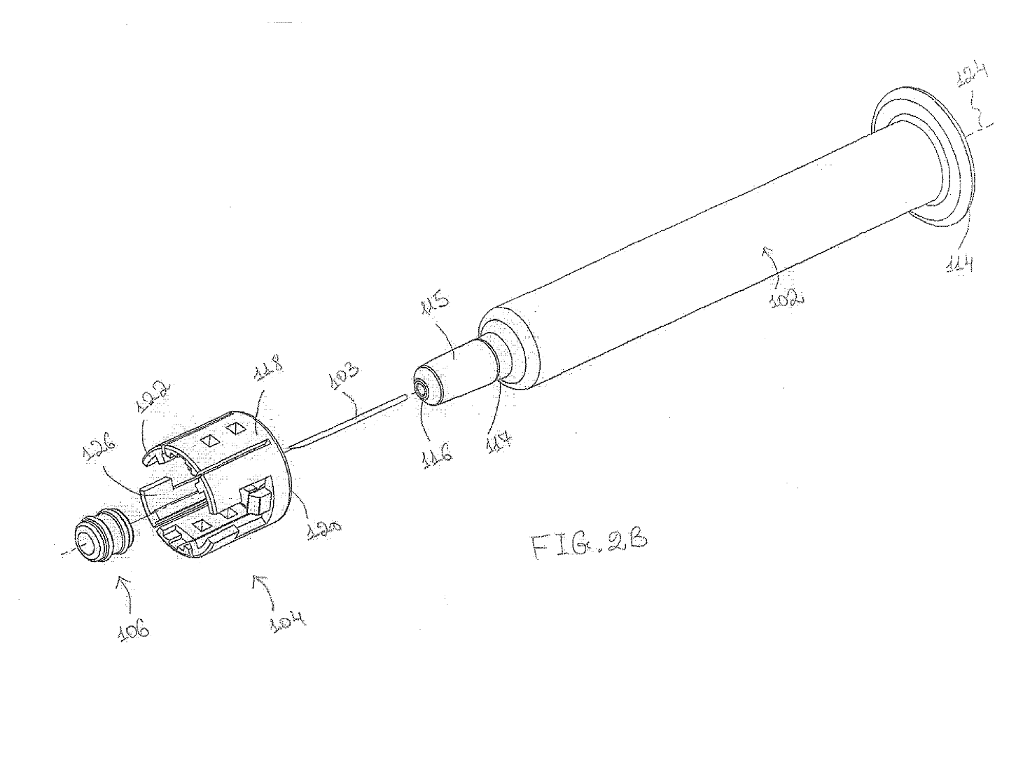

Reference is now made to FIG. 1, which is a simplified pictorial illustration of a medication delivery assembly constructed and operative in accordance with a preferred embodiment of the invention and to FIGS. 2A and 2B, which are simplified respective detailed front and rear sub assembly exploded view illustrations of the medication delivery assembly of FIG. 1.

As seen in FIG. 1, there is provided a medication delivery assembly 100 adapted to fit a standard injection device 102, which may be pre-filled with medication. The medication delivery assembly 100 may be alternatively adapted to fit a pen injector.

FIGS. 2A and 2B are exploded view illustrations of the medication delivery assembly 100 including the pre-filled injection device 102, which may contain a medication. The pre-filled injection device 102 having two opposing ends, rearward end 114 and forward end 116 that defines the outlet port of the injection device, while a hollow penetrating element, such as a needle 103 may be attached to the outlet port 116 of the pre-filled injection device 102. The pre-filled injection device 102 and the needle 103 are arranged along a mutual longitudinal axis 124.

The pre-filled injection device having a luer portion 115 at its outlet port 116 and a rearwardly facing stopping rim 117.

The prefilled injection device 102 with the needle 103 are designed to be attached to the medication delivery assembly 100. It can be seen in FIG. 2 that the medication delivery assembly 100 is a sub assembly comprising a connector 104, septum 106, skin interface element 108, microneedle chip 110 and a shield 112. Alternatively, the injection device 102 may be integrally formed with the connector 104, such as by injection molding.

The connector 104 is defined by a generally cylindrical partially open outer circumference 118 and having two opposite ends, rearward end 120 and forward end 122. The connector 104 is arranged along a longitudinal axis 124 and having an inner circumference 126.

The skin interface element 108 is arranged along a longitudinal axis 124 and having a connector engaging portion 132 and a needle engaging portion 134, a rearward end 128 adjacent the connector engaging portion 132 and a forward end 130 adjacent the needle engaging portion 134. The connector engaging portion 132 of the skin interface element 108 is adapted to be inserted into the connector 104. The microneedle chip 110 is adapted to be coupled to the needle engaging portion end 130 of the skin interface element 108. One side of the skin interface element 108 has a straight surface, which is operative to fit skin surface while injection is performed.

The septum 106 is arranged along a longitudinal axis 124 and is defined by a generally cylindrical outer circumference 136. The septum 106 is adapted to be inserted into the skin interface element 108.

The shield 112 is arranged along a longitudinal axis 124 and having a forwardly facing edge 138 and a rearwardly facing edge 140, which are connected by an outer surface 142. The shield 112 further has locking arms 144 extending partially rearwardly of the rearward edge 140. The shield 112 is adapted to cover the skin interface element 108.

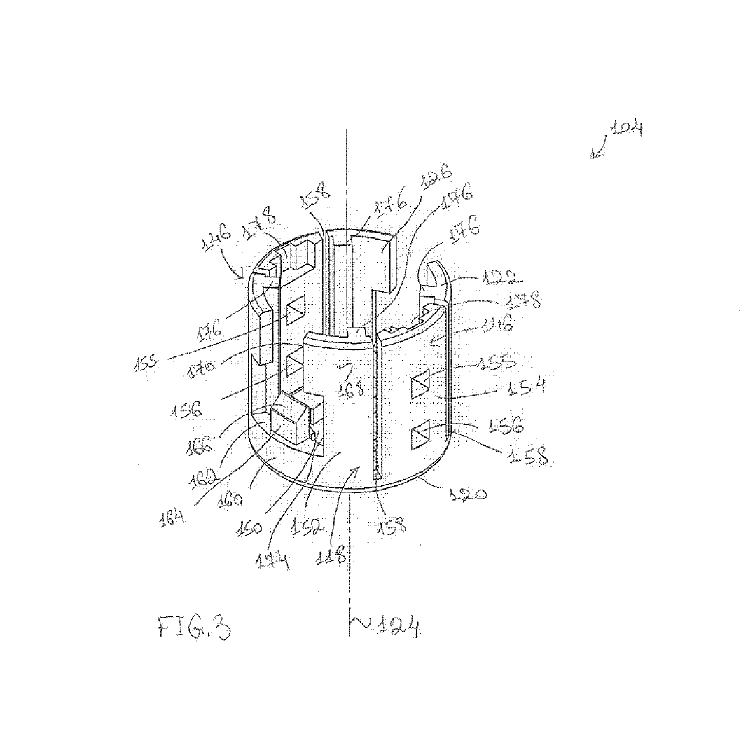

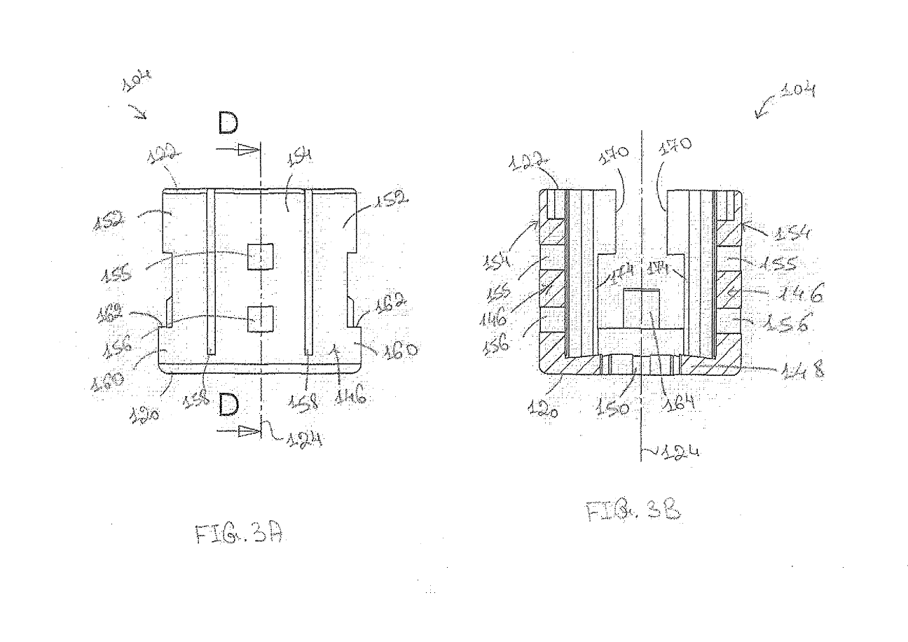

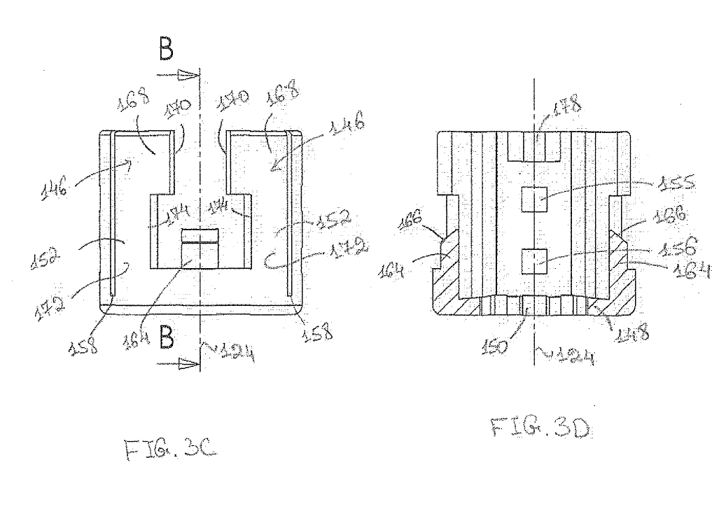

Reference is now made to FIGS. 3, 3A-3D, which illustrate the connector 104 forming part of the medication delivery assembly 100 of FIGS. 1-2B. The connector 104 may be an integrally formed element, preferably formed of plastic, which is symmetric about a longitudinal axis, such as axis 124 (FIGS. 1-2B).

As noted hereinabove with reference to FIGS. 1-2B, the connector 104 is defined by a generally cylindrical partially open outer circumference 118 and having two opposite ends, rearward end 120 and forward end 122. The connector 104 has an inner circumference 126. The connector's 104 outer circumference 118 includes two opposed generally cylindrical engaging arms 146 extending from an annular connecting wall 148 and forming an imaginary cylinder arranged about longitudinal axis 124.

It can be seen on FIGS. 3B and 3D that there is an aperture 150 formed in the connecting wall 148 of the connector 104, acting as a resilient lock for enabling insertion of the pre-filled injection device 102. The aperture is preferably surrounded by a segmented rim.

Each engaging arm 146 has two lateral portions 152 and a medial portion 154 separating between them. There are a forward skin interface element holding recess 155 and a rearward skin interface element holding recess 156 extending through the medial portion 154 of arm 146. There are grooves 158 separating between medial portion 154 and lateral portions 152 of the engaging arms 146, the grooves extend from the outer circumference 118 to the inner circumference 126.

The lateral portions of engaging arm 146 have a forward wide portion 168 defining edge 170 and more narrow portion 172 defining edge 174.

Each lateral portion of engaging arm 146 having a longitudinal rim 176 located adjacent to groove 158, and extending from forward end 122 and the connecting wall 148.

The medial portion 154 of the engaging arm 146 has a stepped recess 178 on its inner circumference 126 and extends rearwardly from forward end 122 partially along the medial portion 154 of the engaging arm 146.

The connector has a raised wall portion 160 connecting between the lateral portions 152 of the engaging arms 146, which has a forwardly facing end 162. There is a raised releasing protrusion 164 located on the forwardly facing wall 162.

Said raised protrusion 164 having an outwardly facing sloped end 166.

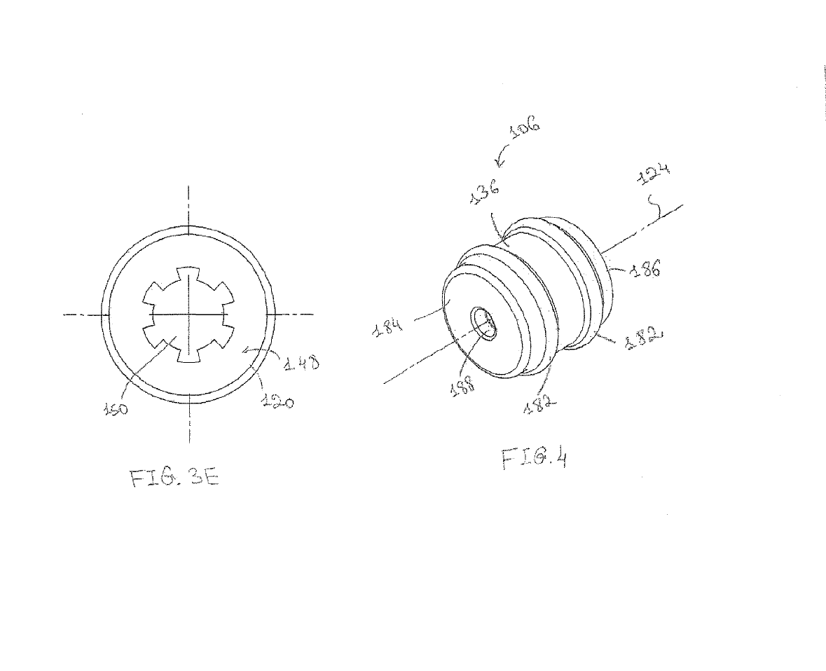

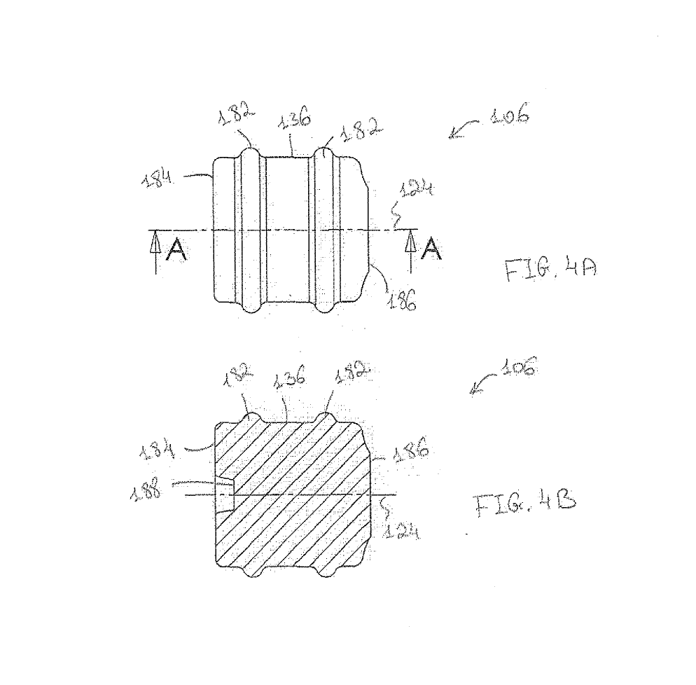

Reference is now made to FIGS. 4, 4A-4C, which illustrate the septum 106 forming part of the medication delivery assembly 100 of FIGS. 1-2B.

The septum 106 may be an integrally formed element, preferably formed of silicon rubber or thermoplastic material with similar characteristics. The septum 106 is symmetric about a longitudinal axis, such as axis 124 (FIGS. 1-2B).

As noted hereinabove with reference to FIGS. 1-2B, the septum 106 is defined by a generally cylindrical outer circumference 136. There are several integrally formed annular rings 182 of a greater diameter than the outer circumference 136. The rings 182 are formed on the outer circumference 136 in a longitudinally spaced manner.

The septum 106 further has two opposite ends, a rearward end 184 and a forward end 186. A longitudinal recess 188 is extending from rearward end 184 partially through the septum 106. The forward end 186 is concave in order to fit tightly within skin interface element 108 and thus prevent or minimize dead space.

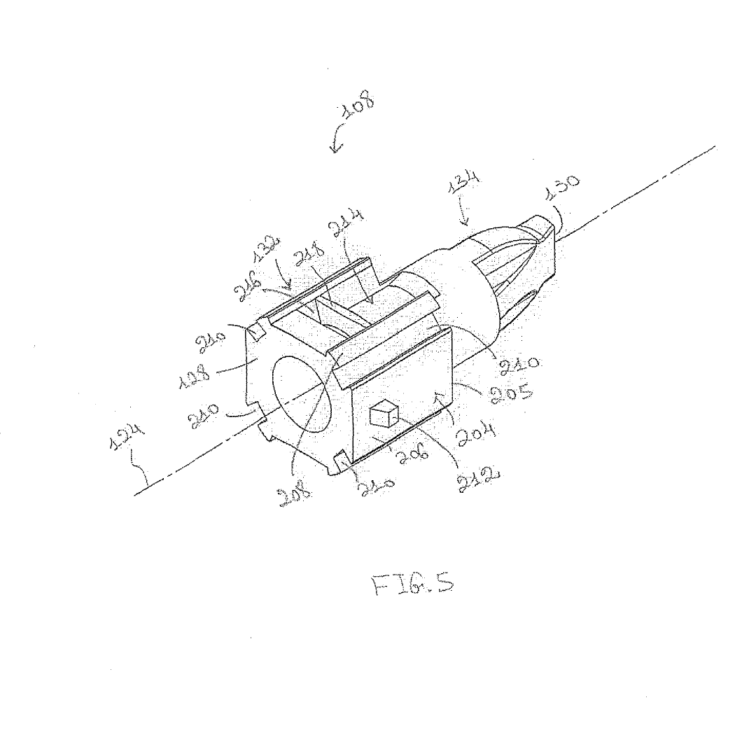

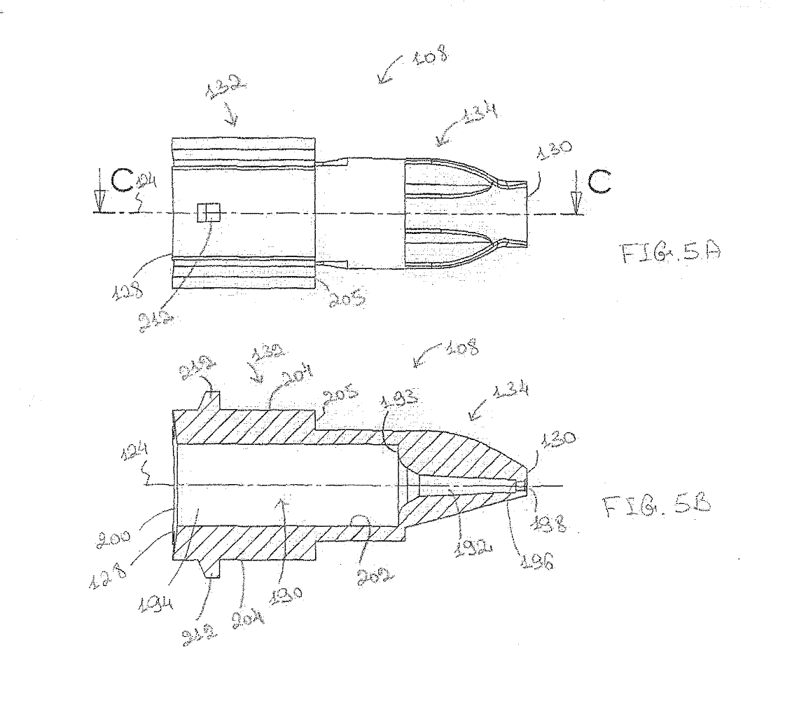

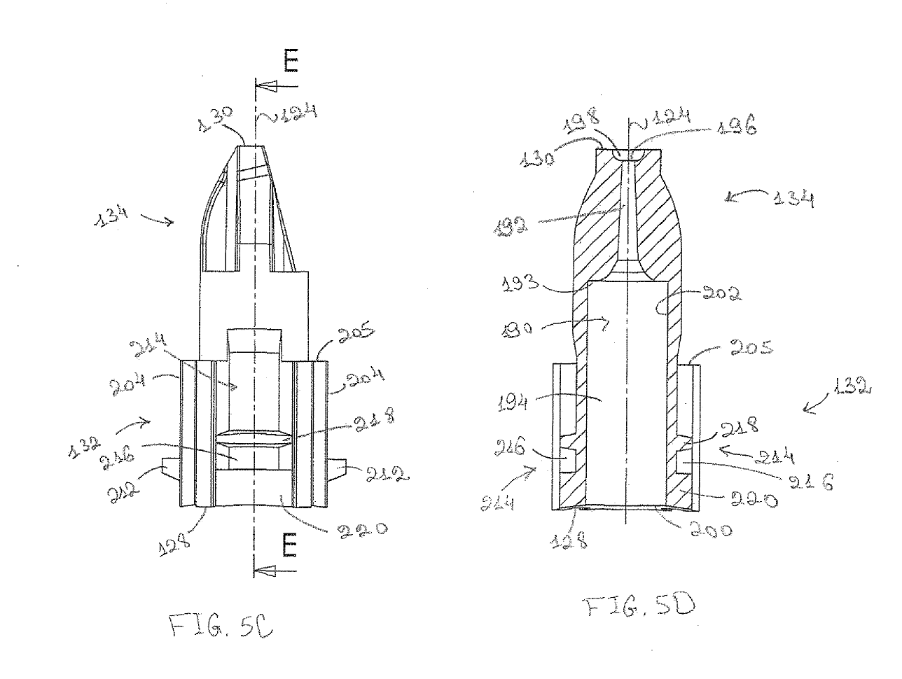

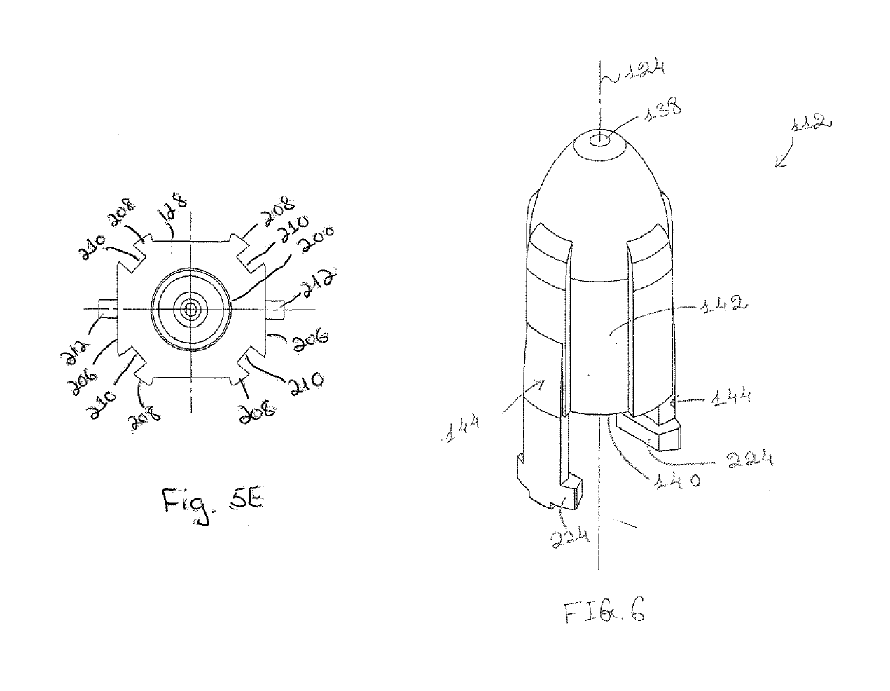

Reference is now made to FIGS. 5, 5A-5E, which illustrate the skin interface element 108 forming part of the medication delivery assembly 100 of FIGS. 1-2B. The skin interface element 108 is an integrally formed element, preferably formed of plastic, which is symmetric about a longitudinal axis, such as axis 124 (FIGS. 1-2B), in all respects other than with respect to the needle engaging portion 134.

As noted hereinabove with reference to FIGS. 1-2B, the skin interface element 108 is arranged along a longitudinal axis 124 and having a connector engaging portion 132 and a needle engaging portion 134, a rearward end 128 adjacent the connector engaging portion 132 and a forward end 130 adjacent the needle engaging portion 134. The connector engaging portion 132 of the skin interface element 108 is adapted to be inserted into the connector 104. The microneedle chip 110 is adapted to be coupled to the needle engaging portion end 130 of the skin interface element 108.

The skin interface element 108 having a flow path 190 therein, comprised of a small diameter forward portion 192 and greater diameter rearward portion 194, forming a shoulder 193 therebetween. The forward portion 192 terminates at flow path forward end 196. There is a recessed area 198 provided between flow path forward end 196 and forward end 130. The rearward portion 194 terminates at flow path rearward end 200. The rearward portion 194 has a generally cylindrical inner surface 202. This specific construction of the flow path 190 is designed to prevent or minimize dead space.

The connector engaging portion 132 having first two opposite faces 204. Each face 204 if formed of a skin interface element medial portion 206 and two laterally spaced portions 208, defining grooves 210 from each side of the medial portion 206, which extend through the entire length of the skin interface element medial portion 132 and the two laterally spaced portions 208. A connector locking protrusion 212 is positioned generally at the rearward portion of the skin interface element medial portion 206. The medial portion 206 defines a forwardly facing edge 205.

The second two opposite faces 214 forming are each forming a shield locking portion 216. The shield locking portion 216 is formed between a forwardly disposed connecting flange 218, which is connecting between laterally spaced portions 208 and between a rearwardly disposed connecting shoulder 220, which is connecting between laterally spaced portions 208 and terminates at rearward end 128.

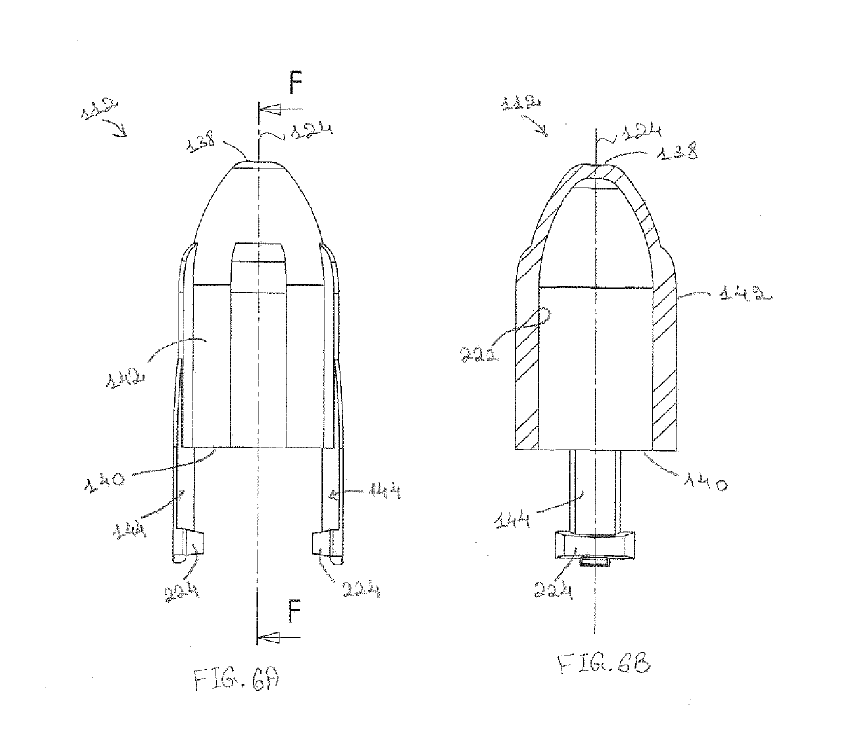

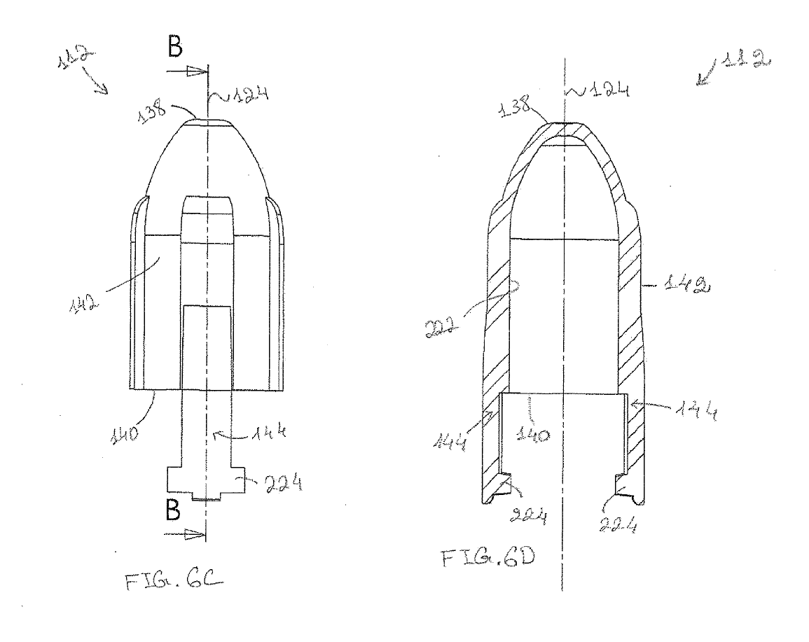

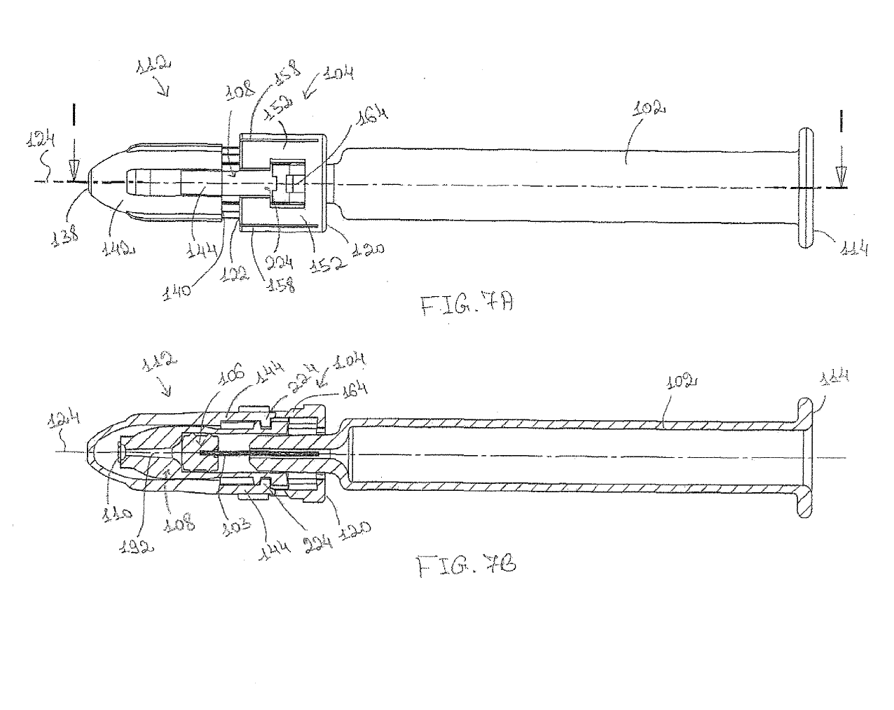

Reference is now made to FIGS. 6, 6A-6D, which illustrate the shield element 112 forming part of the medication delivery assembly 100 of FIGS. 1-2B. The shield element 112 is an integrally formed element, preferably formed of plastic, which is symmetric about a longitudinal axis, such as axis 124 (FIGS. 1-2B).

As noted hereinabove with reference to FIGS. 1-2B, the shield 112 is arranged along a longitudinal axis 124 and having a forwardly facing edge 138 and a rearwardly facing edge 140, which are connected by an outer surface 142. The shield 112 further has locking arms 144 extending partially rearwardly of the rearward edge 140. The shield 112 is adapted to cover the skin interface element 108.

The shield 112 further defines an inner surface 222. The locking arms 144 having an integrally formed, generally rearwardly disposed skin interface element locking protrusions 224, which are generally wider than the locking arms 144. The locking protrusions 224 are extending internally from the outer surface of the locking arms 144.

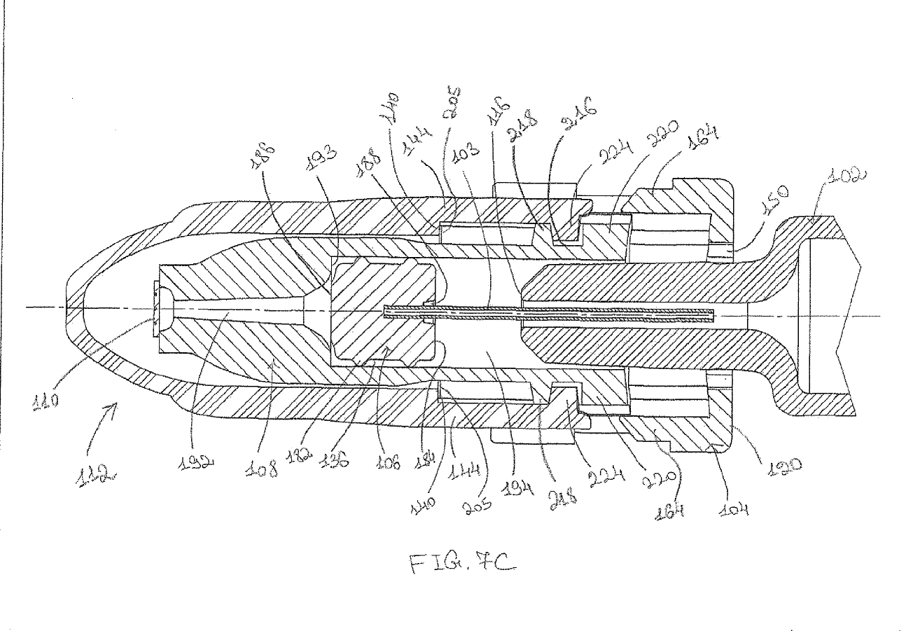

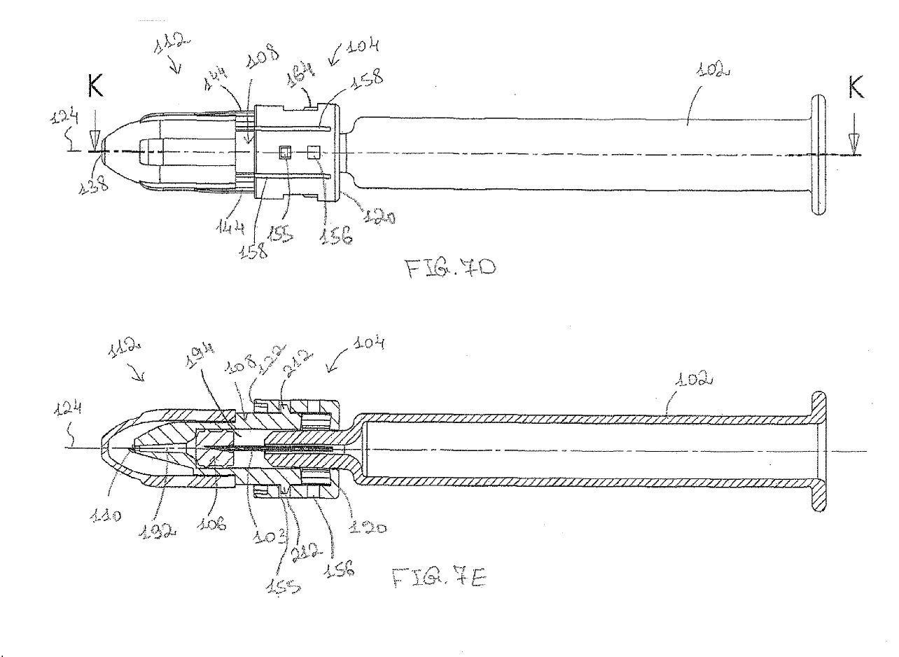

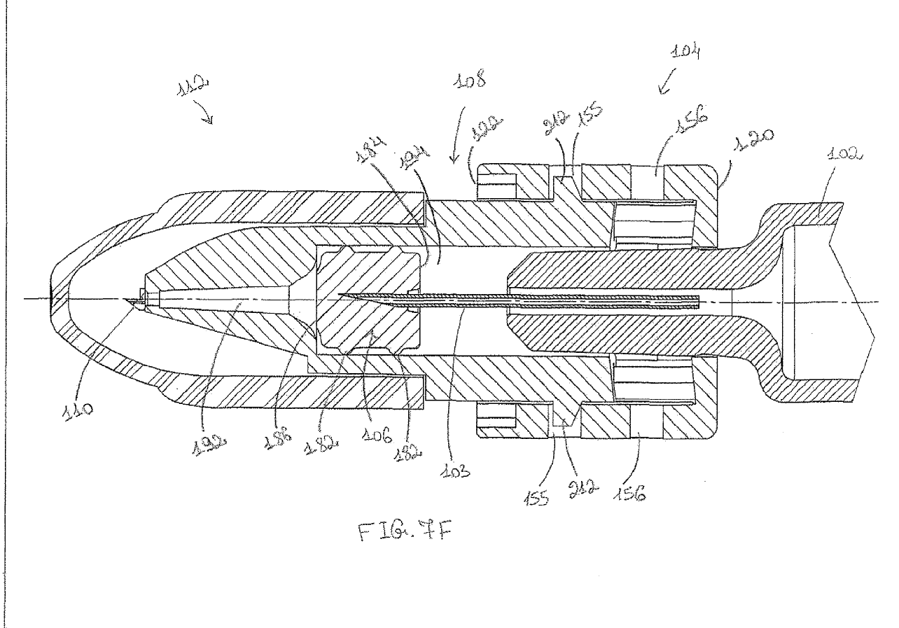

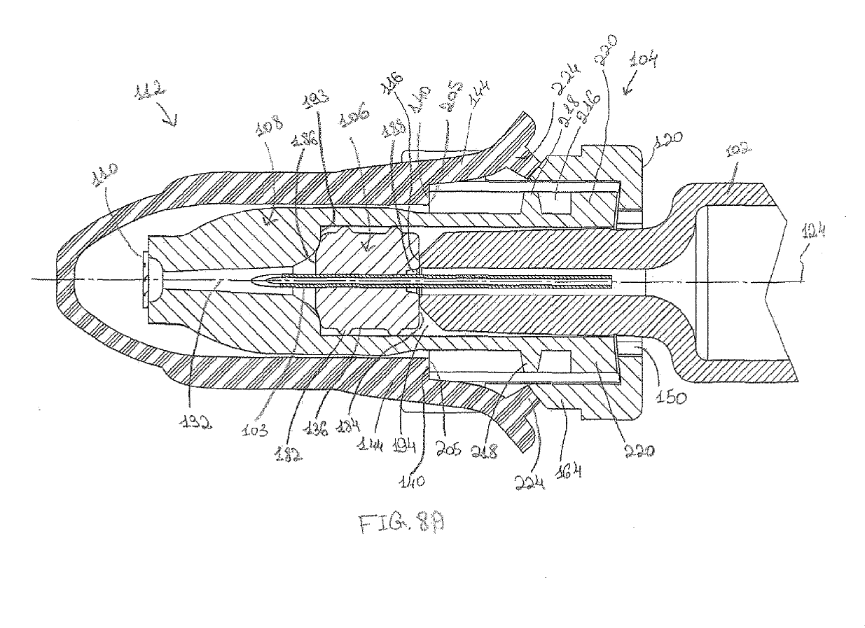

Reference is now made to FIGS. 7A-7F, which are simplified sectional illustrations of the medication delivery assembly 100 of FIG. 1 in an inactive operative position, while in engagement with the prefilled injection device 102.

It can be seen from the above mentioned drawings showing the medication delivery assembly 100 in an inactive position that the medication delivery assembly 100 may be attached to a pre-filled injection device 102. The prefilled injection device 102 may be attached to the connector 104 of medication delivery assembly 100 by means of a stopping rim 117, positioned on the luer portion 115 of the prefilled injection device 102. The luer portion 115 of the injection device 102 is inserted through the aperture 150 of the connector 104. The rim of the aperture 150 is preferably segmented and slightly undersized for the lip of the stopping rim 117, so that the rim of the aperture 150 momentarily flexes outwards as the luer portion 115 is inserted through the aperture 150 of the connector 104 and snaps into place behind the stopping rim 117.

The connector 104 and the injection device 102 then become permanently attached such that they cannot be readily released from each other in a non-destructive manner. It is appreciated that the injection device 102 may be integrally formed with the connector 104, for example by means of injection molding.

It can also be seen from the above mentioned drawings that the skin interface element 108 at the inactive position may be engaged with the connector 104 in a first lockable manner. A connector locking protrusion 212 on the skin interface element 108 is engaged in a lockable manner within the forward skin interface element holding recess 155.

The septum 106 may be located within the skin interface element 108 flow path 190 and may be securely held within by means of annular rings 182 that are frictionally held against the cylindrical inner surface 202. The annular rings 182 may also provide a seal by preventing the fluid from the prefilled injection device 102 that is flowing through the flow path 190 from flowing around the septum 106. The septum is spaced from the luer portion 115 of the prefilled injection device 102.

The sharp end of the needle 103 of the pre-filled injection device 102 is extending into the septum 106 without piercing the septum therethrough at the inactive position. The sharp end of the needle 103 is not exposed in this position, thus fluid flow is not permitted.

The microneedle chip 110 is preferably permanently attached to the forward end 130 of the skin interface element 108.

The shield 112 may be attached to the skin interface element 108 at the inactive position. The rearwardly facing edge 140 of the shield 112 is disposed adjacent to the forwardly facing edge 205 of the skin interface element 108.

It can further be seen that the skin interface element locking protrusions 224 of the shield 112 are fixedly engaged within the shield locking portion 216, due to the fact that the locking arms 144 are held between the faces 214 of the skin interface element 108 and the inner circumference 126 of the connector 104.

It is appreciated that the medication delivery assembly 100 in the state shown in FIGS. 7A-7F is capable of preventing inadvertent microneedle puncturing and disposal of medication by means of shielding the microneedle chip 110 and plugging the needle 103 of the prefilled injection device 102.

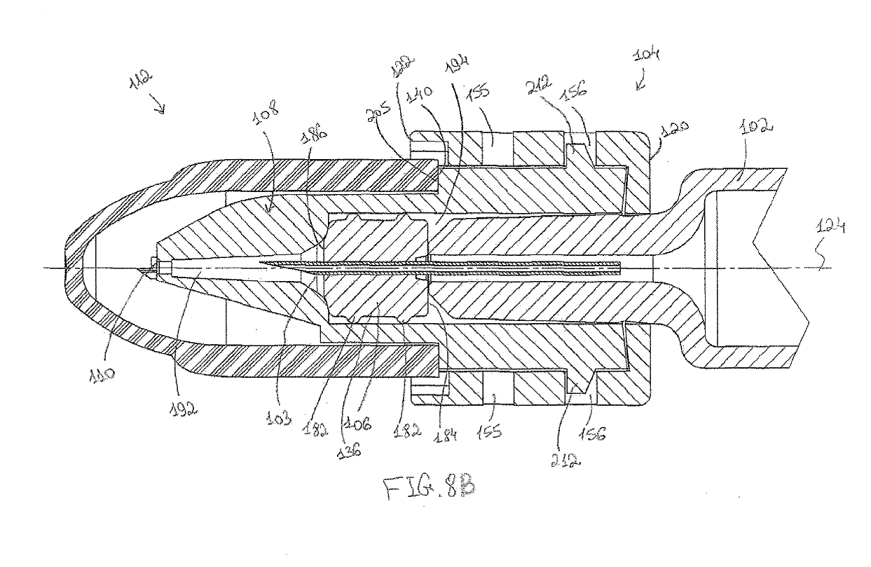

Reference is now made to FIGS. 8A and 8B, which are simplified partial enlargement of sectional illustration of the medication delivery assembly 100 of FIG. 1 in an active operative position, while in engagement with the prefilled injection device 102.

It can be seen from the above mentioned drawings showing the medication delivery assembly 100 in an active position that the medication delivery assembly 100 may be attached to a pre-filled injection device 102. The prefilled injection device 102 may be attached to the connector 104 of medication delivery assembly 100 by means of a stopping rim 117, positioned on the luer portion 115 of the prefilled injection device 102. The luer portion 115 of the injection device 102 is inserted through the aperture 150 of the connector 104. The rim of the aperture 150 is preferably segmented and slightly undersized for the lip of the stopping rim 117, so that the rim of the aperture 150 momentarily flexes outwards as the luer portion 115 is inserted through the aperture 150 of the connector 104 and snaps into place behind the stopping rim 117.

The connector 104 and the injection device 102 then become permanently attached such that they cannot be readily released from each other in a non-destructive manner. It is appreciated that the injection device 102 may be integrally formed with the connector 104, for example by means of injection molding.

It can also be seen from the above mentioned drawings that the skin interface element 108 at the active position is engaged with the connector 104 in a second lockable manner. Following rearward displacement of the shield 112, in order to activate the medication delivery assembly 100, a connector locking protrusion 212 on the skin interface element 108 may be displaced and become engaged in a lockable manner within the rearward skin interface element holding recess 156.

The septum 106 may be located within the skin interface element 108 flow path 190 and may be securely held within by means of annular rings 182 that are frictionally held against the cylindrical inner surface 202. The annular rings 182 are also providing a seal by preventing the fluid from the prefilled injection device 102 that is flowing through the flow path 190 from flowing around the septum 106.

The sharp end of the needle 103 of the prefilled injection device 102 may extend throughout the septum 106 at the active position. The septum rearward end 184 is disposed adjacent the forward end 116 of the prefilled injection device 102. The forward end 116 of the pre-filled injection device 102 may supports the septum 106 and thus prevent rearward movement of the septum 106 due to back pressure of the medication. The sharp end of the needle 103 may be exposed into the forward portion 192 of the flow path 190 of the skin interface element 108 in the active position, thus fluid flow may be permitted from the prefilled injection device 102 via the needle 103, further via the forward portion 192 of the flow path 190 of the skin interface element 108 and through the microneedle array arranged on the microneedle chip 110.

In accordance to a preferred embodiment of the invention, the microneedle chip 110 may be formed of at least one hollow penetrating element, which is implemented as at least one hollow microneedle integrally formed with an underlying substrate.

The microneedle chip 110 may be preferably formed of two hollow microneedles integrally formed with an underlying substrate or may be alternatively formed of a linear array of at least three hollow microneedles integrally formed with an underlying substrate.

Each microneedle within the microneedle chip 110 may be preferably formed primarily from silicon.

It may be appreciated that in a particular embodiment of the invention, each hollow microneedle is formed with at least one upright surface standing upright relative to a surface of said underlying substrate, an inclined surface intersecting said at least one upright surface and a fluid flow bore intersecting said inclined surface.

In accordance to an embodiment of the invention, each hollow microneedle is preferably less than 1 mm of height.

Each hollow microneedle is located adjacent to an edge of said underlying substrate in such a manner that the microneedle having a height, and being less than twice its own height away from the edge.

It is further appreciated that the microneedle chip 110 may be constructed as it is previously disclosed in U.S. Pat. Nos. 7,648,484 and 6,533,949, assigned to Nanopass Technologies.

The microneedle chip 110 may be permanently attached to the forward end 130 of the skin interface element 108.

In active position, the shield 112 may be disposed over the skin interface element 108, however it is no longer attached to the skin interface element 108. The rearwardly facing edge 140 of the shield 112 is still disposed adjacent to the forwardly facing edge 205 of the skin interface element 108 in the active position.

It can further be seen that the skin interface element locking protrusions 224 of the shield 112 are no longer engaged within the shield locking portion 216. Due to manual rearward displacement of the shield 112, the skin interface element 108 is adapted to be displaced rearwardly as well, the connector locking protrusion 212 of the skin interface element 108 is enabled to move out of engagement with the forward skin interface element holding recess 155 of the connector 104 and becomes instead locked within the rearward holding recess 156 of the connector 104. The locking of the connector locking protrusion 212 with the holding recess 156 is made permanent due to the structure of the locking protrusions 212, which have one straight end and one sloped end, such that the connector 104 and the skin interface element 108 cannot be unlocked unless sufficient force is exerted to overcome this locking relation that is not readily achieved manually.

Simultaneously, due to the rearward movement of the skin interface element 108, the locking arms 144 of the shield 112 are deflected outwardly and sliding generally rearwardly over the sloped end 166 of the raised protrusion 164 of the connector 104.

The rearward end 128 of the skin interface element 108 is positioned adjacent the connecting wall 148 of the connector 104 at the active position.

It is appreciated that the medication delivery assembly 100 in the state shown in FIGS. 8A and 8B is a transitional stage of activation, which still doesn't allow inadvertent microneedle puncturing, however the shield 112 is released from lockable engagement at this stage and is ready to be removed from the medication delivery assembly 100 and the hollow needle 103 penetrates entirely through the septum 106.

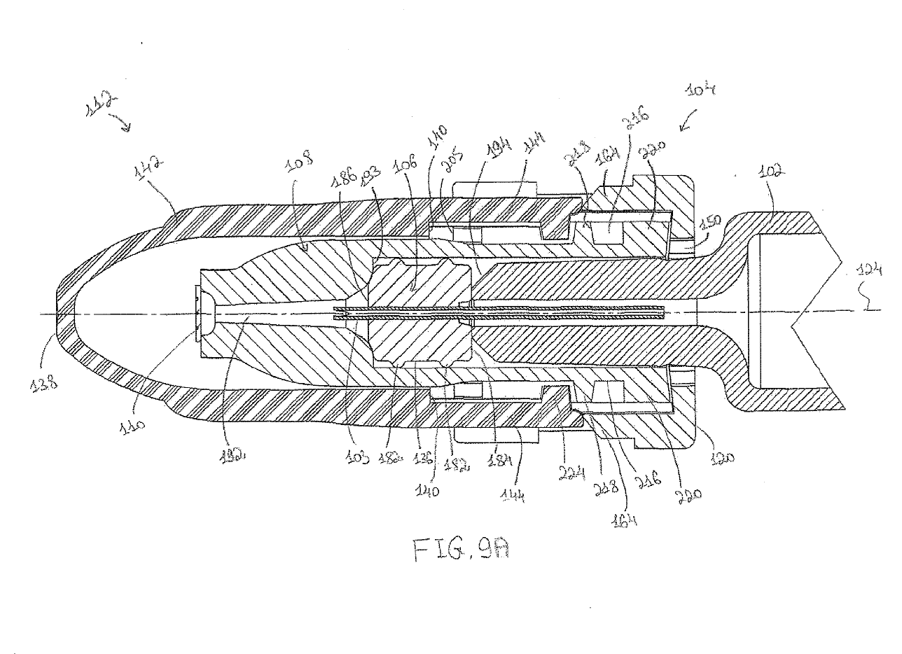

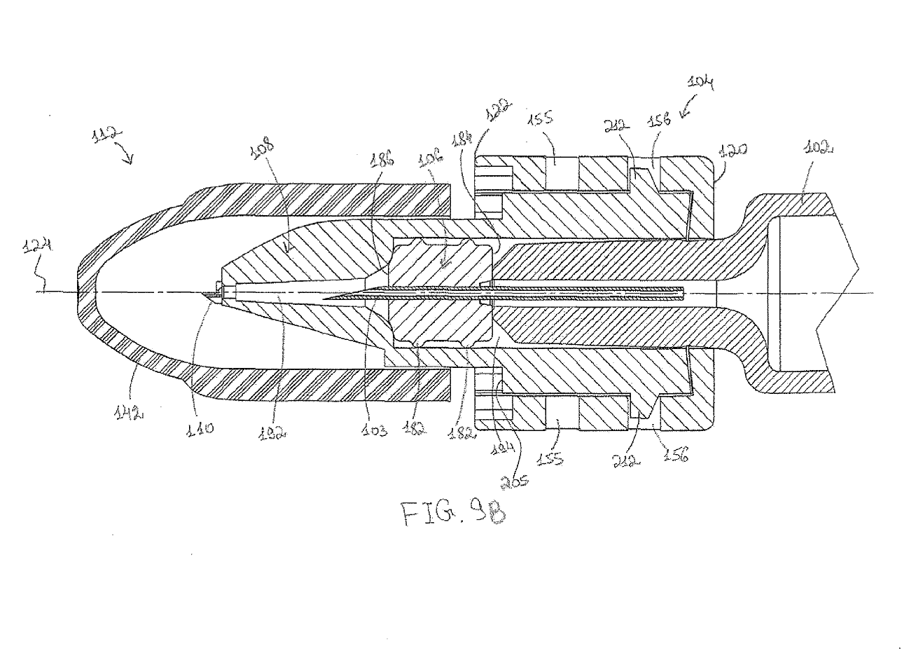

Reference is now made to FIGS. 9A and 9B, which are simplified partial enlargement of sectional illustration of the medication delivery assembly 100 of FIG. 1 in an open operative position, while in engagement with the prefilled injection device 102.

It can be seen from the above mentioned drawings showing the medication delivery assembly 100 in an open position that the medication delivery assembly 100 is attached to a pre-filled injection device 102. The prefilled injection device 102 may be attached to the connector 104 of medication delivery assembly 100 by means of a stopping rim 117, positioned on the luer portion 115 of the prefilled injection device 102. The luer portion 115 of the injection device 102 is inserted through the aperture 150 of the connector 104. The rim of the aperture 150 is preferably segmented and slightly undersized for the lip of the stopping rim 117, so that the rim of the aperture 150 momentarily flexes outwards as the luer portion 115 is inserted through the aperture 150 of the connector 104 and snaps into place behind the stopping rim 117.

The connector 104 and the injection device 102 then become permanently attached such that they cannot be readily released from each other in a non-destructive manner. It is appreciated that the injection device 102 may be integrally formed with the connector 104, for example by means of injection molding.

It can also be seen from the above mentioned drawings that the skin interface element 108 at the open stage is engaged with the connector 104 in a second lockable manner.

As previously shown in FIGS. 8A and 8B, following rearward displacement of the shield 112, in order to activate the medication delivery assembly 100, a connector locking protrusion 212 on the skin interface element 108 is displaced and engaged in a lockable manner within the rearward skin interface element holding recess 156.

In an open operative position shown in FIGS. 9A and 9B, the shield 112 is still disposed over the skin interface element 108.

The shield 112 may be released from rearward displacement and consequentially the locking arms 144 of the shield 112 are not deflected anymore, thus the locking arms 144 return to their normal position while sliding generally forwardly along the sloped end 166 of the raised protrusion 164 of the connector 104. The locking arms 144 in the open operative position are disposed between the faces 214 of the skin interface element 108 and between the inner circumferences 126 of the connector 104, however the locking arms 144 are not held at this position, as it was shown on FIGS. 7A-7F in the inactive position, since the skin interface element locking protrusions 224 of the shield 112 are out of engagement with the shield locking portion 216 following the activation stage, as described with reference to FIGS. 8A and 8B.

While referring specifically to FIGS. 9A and 9B, the rearwardly facing edge 140 of the shield 112 is spaced from the forwardly facing edge 205 of the skin interface element 108 as the shield 112 is moving forwardly.

The skin interface element 108 at the open operative stage is displaced rearwardly, the connector locking protrusions 212 of the skin interface element 108 are engaged with the rearward skin interface element holding recess 156 of the connector 104.

The septum 106 is located within the skin interface element 108 flow path 190 and is securely held within by means of annular rings 182 that are frictionally held against the cylindrical inner surface 202. The annular rings 182 are also providing a seal by preventing the fluid from the prefilled injection device 102 that is flowing through the flow path 190 from flowing around the septum 106.

The sharp end of the needle 103 of the prefilled injection device 102 extends throughout the septum 106 at the open operative position. The septum rearward end 184 is disposed adjacent the forward end 116 of the prefilled injection device 102. The sharp end of the needle 103 is exposed into the forward portion 192 of the flow path 190 of the skin interface element 108 in the open operative position, thus fluid flow is permitted from the prefilled injection device 102 via the needle 103, further via the forward portion 192 of the flow path 190 of the skin interface element 108 and through the microneedle array arranged on the microneedle chip 110.

In accordance to a preferred embodiment of the invention, the microneedle chip 110 may be formed of at least one hollow penetrating element, which is implemented as at least one hollow microneedle integrally formed with an underlying substrate.

The microneedle chip 110 may be preferably formed of two hollow microneedles integrally fowled with an underlying substrate or may be alternatively formed of a linear array of at least three hollow microneedles integrally formed with an underlying substrate.

Each microneedle within the microneedle chip 110 may be preferably formed primarily from silicon.

It may be appreciated that in a particular embodiment of the invention, each hollow microneedle is formed with at least one upright surface standing upright relative to a surface of said underlying substrate, an inclined surface intersecting said at least one upright surface and a fluid flow bore intersecting said inclined surface.

In accordance to an embodiment of the invention, each hollow microneedle is preferably less than 1 mm of height.

Each hollow microneedle is located adjacent to an edge of said underlying substrate in such a manner that the microneedle having a height, and being less than twice its own height away from the edge.

It is further appreciated that the microneedle chip 110 may be constructed as it is previously disclosed in U.S. Pat. Nos. 7,648,484 and 6,533,949, assigned to Nanopass Technologies.

The microneedle chip 110 is permanently attached to the forward end 130 of the skin interface element 108.

The rearward end 128 of the skin interface element 108 is fixedly positioned adjacent the connecting wall 148 of the connector 104 at the open operative position.

It is appreciated that the medication delivery assembly 100 in the state shown in FIGS. 9A and 9B is a transitional stage of releasing the shield 112, which still doesn't allow inadvertent microneedle puncturing.

Reference is now made to FIGS. 8A and 8B, which are simplified partial enlargement of sectional illustration of the medication delivery assembly 100 of FIG. 1 in an injection position, while in engagement with the prefilled injection device 102.

It can be seen from the above mentioned drawings showing the medication delivery assembly 100 in an injection position that the medication delivery assembly 100 may be attached to a pre-filled injection device 102. The prefilled injection device 102 may be attached to the connector 104 of medication delivery assembly 100 by means of a stopping rim 117, positioned on the luer portion 115 of the prefilled injection device 102. The luer portion 115 of the injection device 102 is inserted through the aperture 150 of the connector 104. The rim of the aperture 150 is preferably segmented and slightly undersized for the lip of the stopping rim 117, so that the rim of the aperture 150 momentarily flexes outwards as the luer portion 115 is inserted through the aperture 150 of the connector 104 and snaps into place behind the stopping rim 117.

The connector 104 and the injection device 102 then become permanently attached such that they cannot be readily released from each other in a non-destructive manner. It is appreciated that the injection device 102 may be integrally formed with the connector 104, for example by means of injection molding.

It can also be seen from the above mentioned drawings that the skin interface element 108 at the injection stage is fixedly engaged with the connector 104 in a second lockable manner. As previously shown in FIGS. 8A and 8B, following rearward displacement of the shield 112, in order to activate the medication delivery assembly 100, a connector locking protrusion 212 on the skin interface element 108 is displaced and engaged in a lockable manner within the rearward skin interface element holding recess 156.

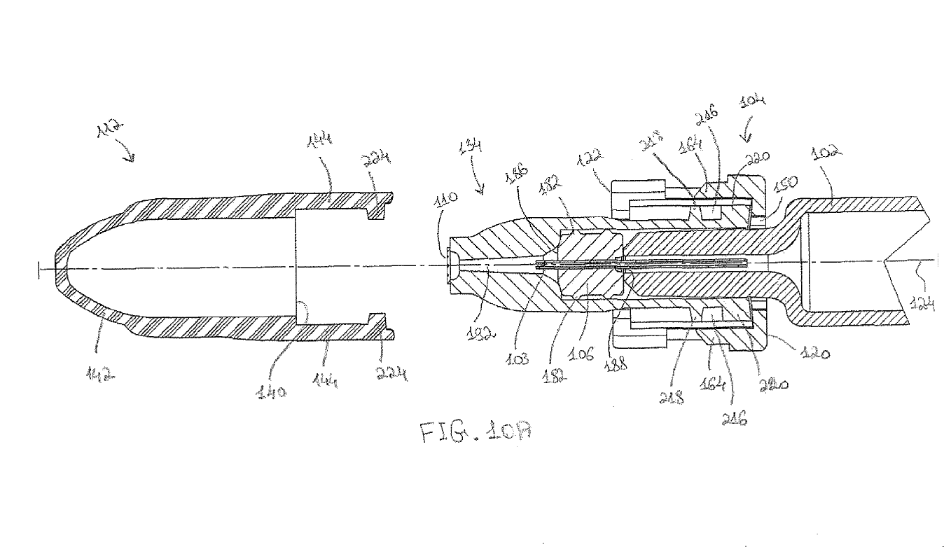

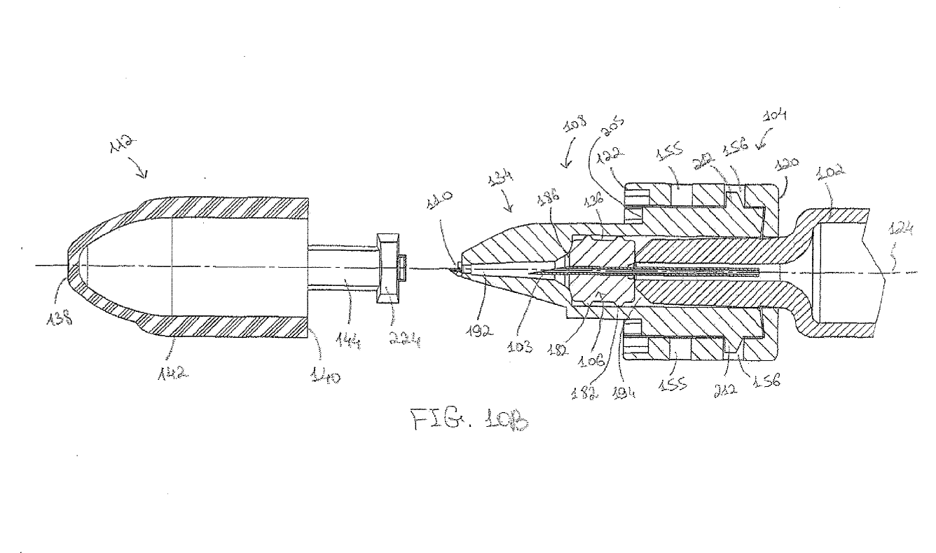

In the injection position shown in FIGS. 10A and 10B, the shield 112 is removed completely from the skin interface element 108.

The skin interface element 108 at the injection position is disposed rearwardly, the connector locking protrusion 212 of the skin interface element 108 are engaged with the rearward skin interface element holding recess 156 of the connector 104.

The septum 106 may be located within the skin interface element 108 flow path 190 and may be securely held within by means of annular rings 182 that are frictionally held against the cylindrical inner surface 202. The annular rings 182 are also providing a seal by preventing the fluid from the prefilled injection device 102 that is flowing through the flow path 190 from flowing around the septum 106.

The sharp end of the needle 103 of the prefilled injection device 102 extends throughout the septum 106 at the injection position. The septum rearward end 184 is disposed adjacent the forward end 116 of the prefilled injection device 102. The sharp end of the needle 103 is exposed into the forward portion 192 of the flow path 190 of the skin interface element 108 in the injection operative position, thus fluid flow is permitted from the prefilled injection device 102 via the needle 103, further via the forward portion 192 of the flow path 190 of the skin interface element 108 and through the microneedle array arranged on the microneedle chip 110.

In accordance to a preferred embodiment of the invention, the microneedle chip 110 may be formed of at least one hollow penetrating element, which is implemented as at least one hollow microneedle integrally formed with an underlying substrate.

The microneedle chip 110 may be preferably formed of two hollow microneedles integrally formed with an underlying substrate or may be alternatively formed of a linear array of at least three hollow microneedles integrally formed with an underlying substrate.

Each microneedle within the microneedle chip 110 may be preferably formed primarily from silicon.

It may be appreciated that in a particular embodiment of the invention, each hollow microneedle is fanned with at least one upright surface standing upright relative to a surface of said underlying substrate, an inclined surface intersecting said at least one upright surface and a fluid flow bore intersecting said inclined surface.

In accordance to an embodiment of the invention, each hollow microneedle is preferably less than 1 mm of height.

Each hollow microneedle is located adjacent to an edge of said underlying substrate in such a manner that the microneedle having a height, and being less than twice its own height away from the edge.

It is further appreciated that the microneedle chip 110 may be constructed as it is previously disclosed in U.S. Pat. Nos. 7,648,484 and 6,533,949, assigned to Nanopass Technologies.

The microneedle chip 110 may be permanently attached to the forward end 130 of the skin interface element 108.

The rearward end 128 of the skin interface element 108 may be fixedly positioned adjacent the connecting wall 148 of the connector 104 at the injection position.

It is appreciated that the medication delivery assembly 100 in the state shown in FIGS. 10A and 10B is an injection stage while the shield 112 is completely removed and injection of fluid from the prefilled injection device 102 throughout the medication delivery assembly 100 and through the microneedles 110 is permitted.

It is further appreciated that following the injection, the shield 112 may be placed back onto the skin interface element 108 as it is shown on FIGS. 9A and 9B. At this position, the shield 112 covers the microneedle chip 110, which provides for safety functionality by preventing inadvertent needle puncturing at the discarding stage.

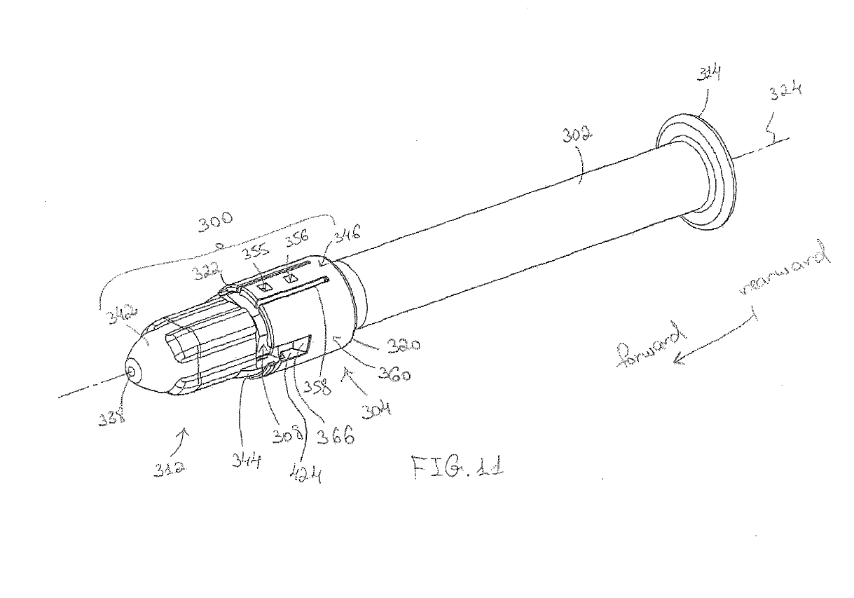

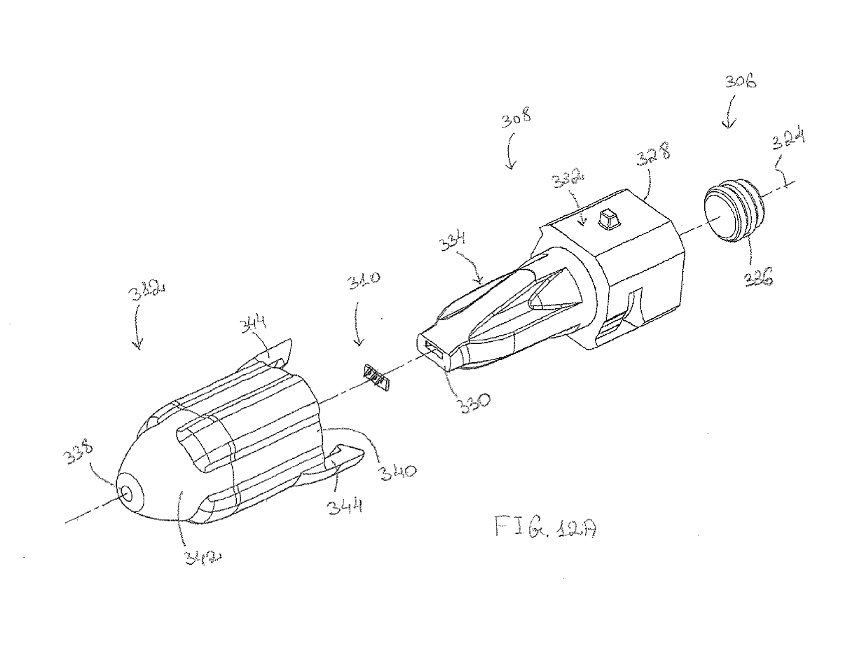

Reference is now made to FIG. 11, which is a simplified pictorial illustration of a medication delivery assembly constructed and operative in accordance with another preferred embodiment of the invention and to FIGS. 12A and 12B, which are simplified respective detailed front and rear sub assembly exploded views illustrations of the medication delivery assembly of FIG. 11.

As seen in FIG. 11, there is provided a medication delivery assembly 300 adapted to fit a standard pre-filled injection device 302.

FIGS. 12A and 12B are exploded view illustrations of the medication delivery assembly 300 including the pre-filled injection device 302, which may contain a medication. The pre-filled injection device 302 having two opposing ends, rearward end 314 and forward end 316 that defines the outlet port of the injection device, while a hollow penetrating element, such as a needle 303 may be attached to the outlet port 316 of the pre-filled injection device 302. The pre-filled injection device 302 and the needle 303 are arranged along a mutual longitudinal axis 324.

The pre-filled injection device having a luer portion 315 at its outlet port 316 and a rearwardly facing stopping rim 317.

The prefilled injection device 302 with the needle 303 are designed to be attached to the medication delivery assembly 300. It can be seen on FIG. 11 that the medication delivery assembly 300 is a sub assembly comprising a connector 304, septum 306, skin interface element 308, microneedle chip 310 and a shield 312.

The connector 304 is defined by a generally cylindrical partially open outer circumference 318 and having two opposite ends, rearward end 320 and forward end 322. The connector 304 is arranged along a longitudinal axis 324 and having an inner circumference 326.

The skin interface element 308 is arranged along a longitudinal axis 324 and having a connector engaging portion 332 and a needle engaging portion 334, a rearward end 328 adjacent the connector engaging portion 332 and a forward end 330 adjacent the needle engaging portion 334. The connector engaging portion 332 of the skin interface element 308 is adapted to be inserted into the connector 304. The microneedle chip 310 is adapted to be coupled to the needle engaging portion end 330 of the skin interface element 308.

The septum 306 is arranged along a longitudinal axis 324 and is defined by a generally cylindrical outer circumference 336. The septum 306 is adapted to be inserted into the skin interface element 308.

The shield 312 is arranged along a longitudinal axis 324 and having a forwardly facing edge 338 and a rearwardly facing edge 340, which are connected by an outer surface 342. The shield 312 further has locking arms 344 extending partially rearwardly of the rearward edge 340. The shield 312 is adapted to cover the skin interface element 308.

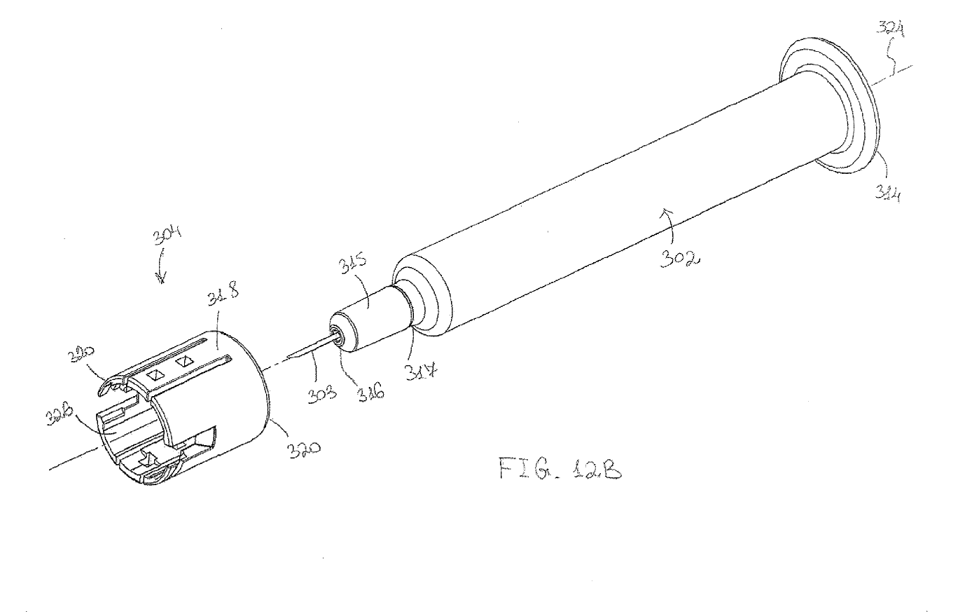

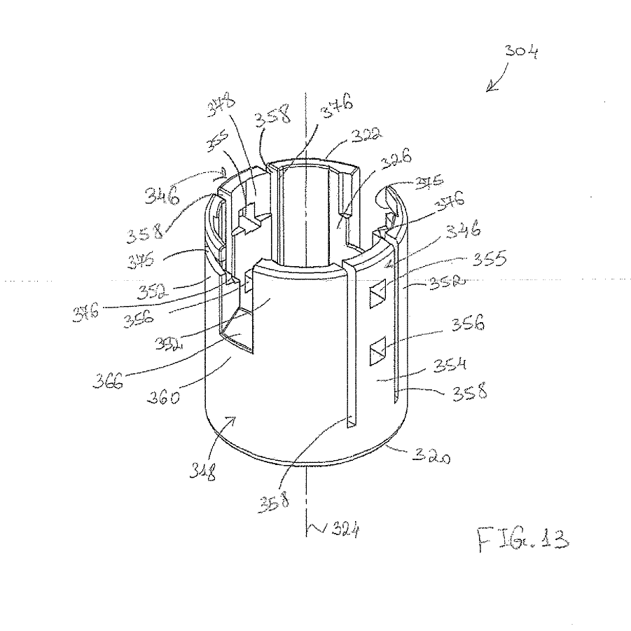

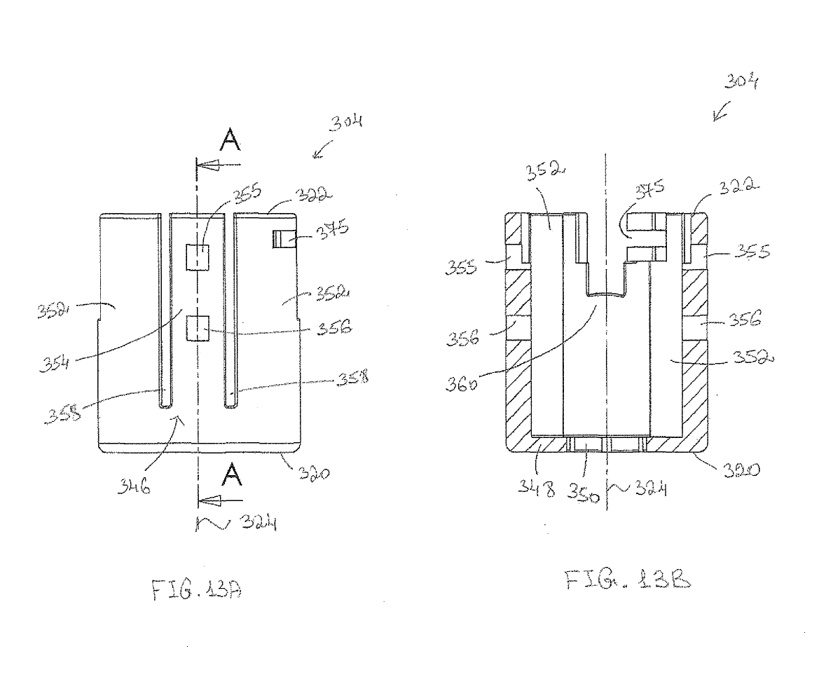

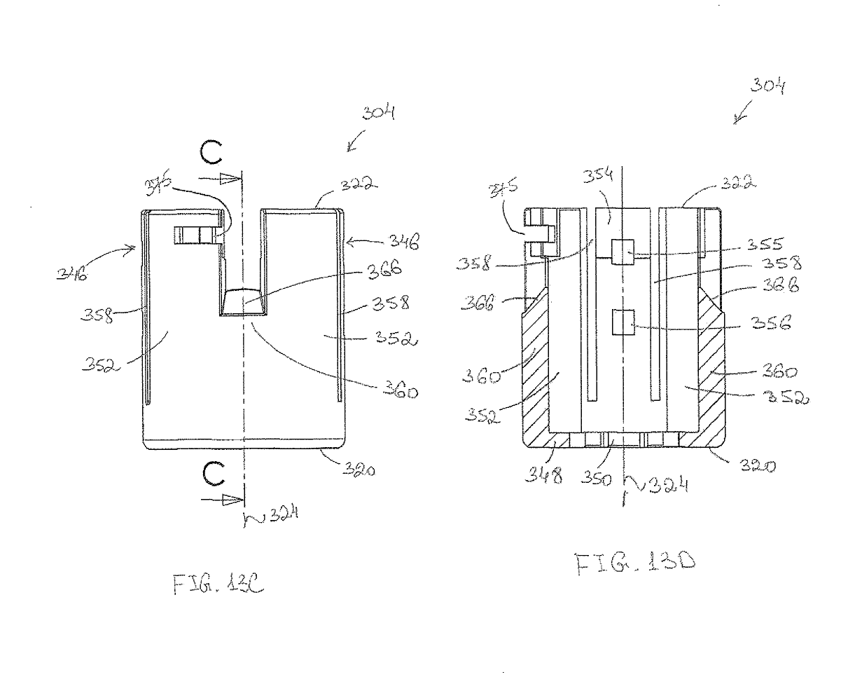

Reference is now made to FIGS. 13, 13A-13E, which illustrate the connector 304 forming part of the medication delivery assembly 300 of FIGS. 11-12B. The connector 304 is an integrally formed element, preferably formed of plastic, which is generally symmetric about a longitudinal axis, such as axis 324 (FIGS. 11-12B), however having an asymmetric feature in order to define assembling direction.

As noted hereinabove with reference to FIGS. 11-12B, the connector 304 may be defined by a generally cylindrical partially open outer circumference 318 and having two opposite ends, rearward end 320 and forward end 322. The connector 304 has an inner circumference 326. The connector's 304 outer circumference 318 includes two opposed generally cylindrical engaging arms 346 extending from an annular connecting wall 348 and forming an imaginary cylinder arranged about longitudinal axis 324.

It can be seen in FIGS. 13B and 13D that there is an aperture 350 formed in the connecting wall 348 of the connector 304, acting as a resilient lock for enabling insertion of the pre-filled injection device 302. The aperture 35 is preferably surrounded by a segmented rim.

Each engaging arm 346 has two lateral portions 352 and a medial portion 354 separating between them. There are a forward skin interface element holding recess 355 and a rearward skin interface element holding recess 356 extending through the medial portion 354 of arm 346. There are grooves 358 separating between medial portion 354 and lateral portions 352 of the engaging arms 346, the grooves extend from the outer circumference 318 to the inner circumference 326.

One of each couple of lateral portions 352 of the engaging arms 346 having a radial skin interface element engaging recess 375 which is extending partially through the circumference of the lateral portion 352. A forwardly facing skin interface element engaging protrusion 376 may be disposed rearwardly of the skin interface element engaging recess 375, extending through the same circumference extent as the skin interface element engaging recess 375.

The medial portion 354 of the engaging arm 346 has a stepped recess 378 on its inner circumference 326 and extends rearwardly from forward end 322 partially along the medial portion 354 of the engaging arm 346.

The connector has a raised wall portion 360 connecting between the lateral portions 352 of the engaging arms 346, which has an outwardly facing sloped end 366.

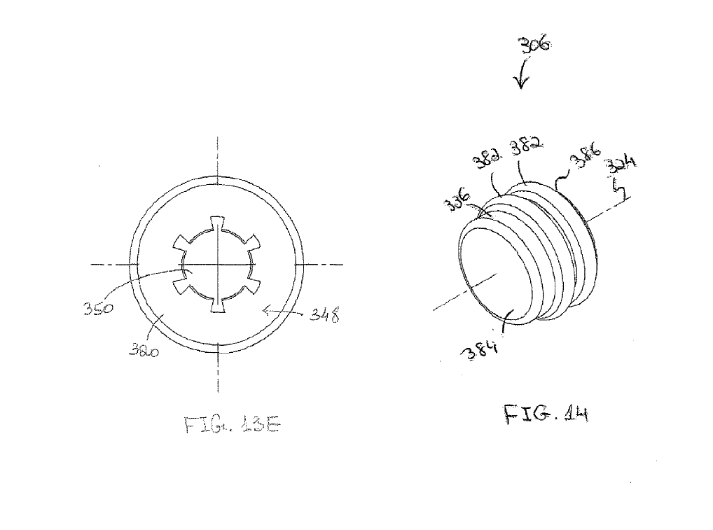

Reference is now made to FIGS. 14, 14A-14C, which illustrate the septum 306 forming part of the medication delivery assembly 300 of FIGS. 11-12B.

The septum 306 may be an integrally formed element, preferably formed of silicon rubber or thermoplastic material with similar characteristics. The septum 106 is preferably symmetric about a longitudinal axis, such as axis 324 (FIGS. 11-12B).

As noted hereinabove with reference to FIGS. 11-12B, the septum 306 may be defined by a generally cylindrical outer circumference 336. There are several integrally formed annular rings 382 of a greater diameter than the outer circumference 336. The rings 382 are formed on the outer circumference 336 in a longitudinally spaced manner. The septum further has two opposite ends, a rearward end 384 and a forward end 386.

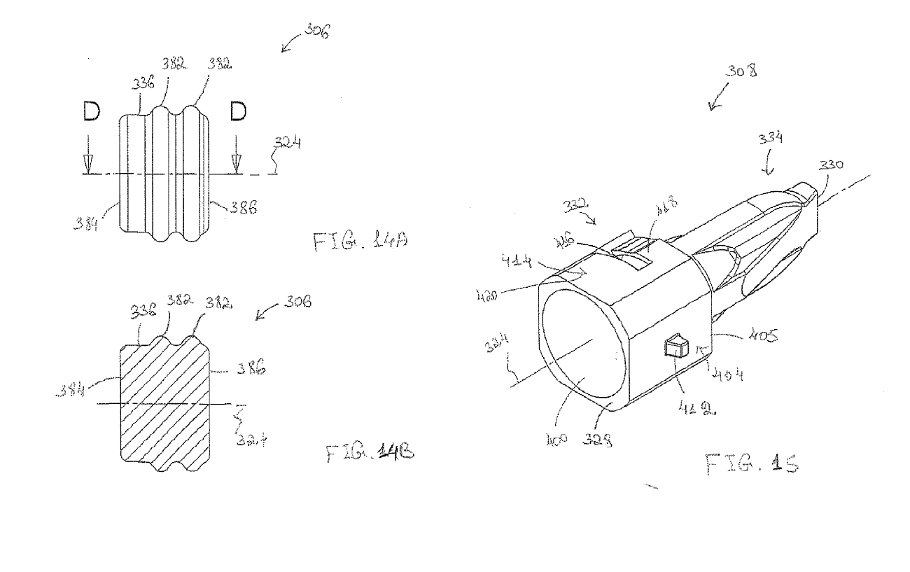

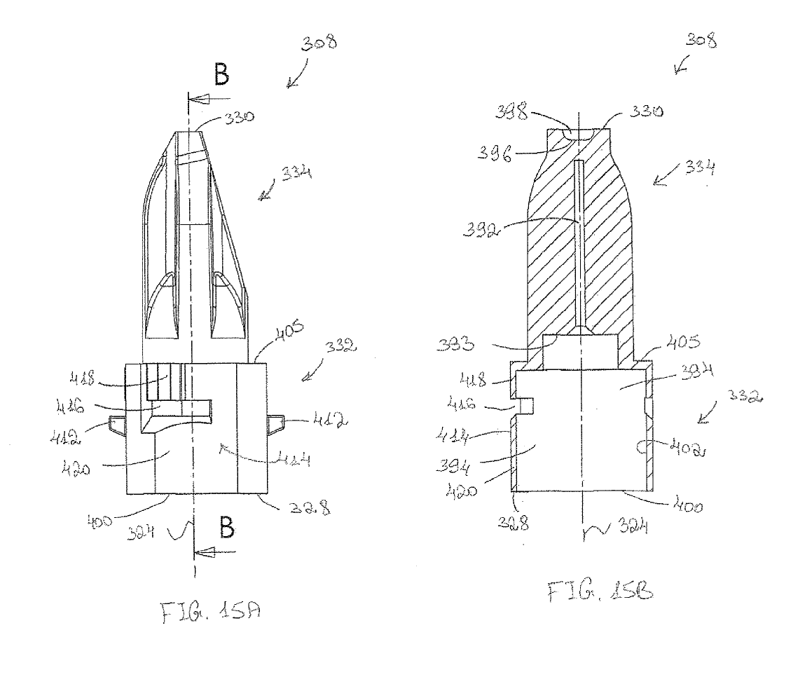

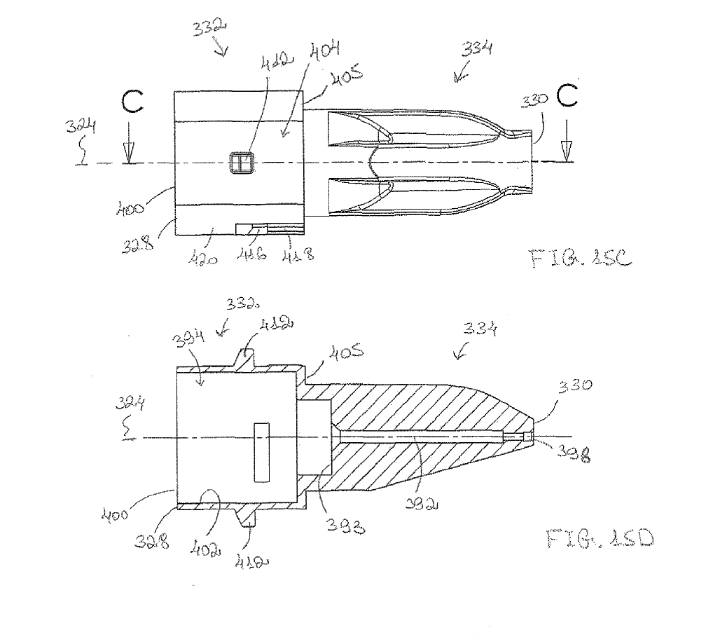

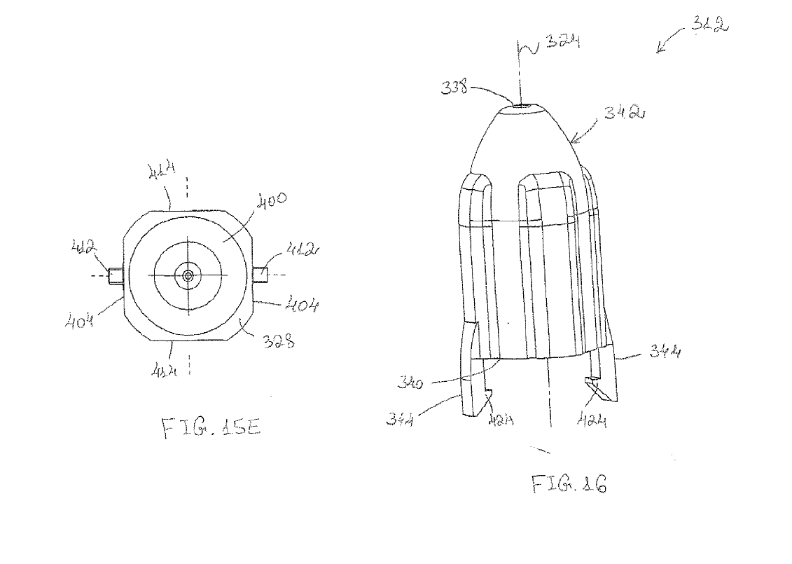

Reference is now made to FIGS. 15, 15A-15E, which illustrate the skin interface element 308 forming part of the medication delivery assembly 300 of FIGS. 11-12B. The skin interface element 308 is preferably an integrally formed element, preferably fowled of plastic, which is generally symmetric about a longitudinal axis, such as axis 324 (FIGS. 11-12B), however having an asymmetric feature in order to define assembling direction.

As noted hereinabove with reference to FIGS. 11-12B, the skin interface element 308 may be arranged along a longitudinal axis 324 and having a connector engaging portion 332 and a needle engaging portion 334, a rearward end 328 adjacent the connector engaging portion 332 and a forward end 330 adjacent the needle engaging portion 334. The connector engaging portion 332 of the skin interface element 308 is adapted to be inserted into the connector 304. The microneedle chip 310 is adapted to be coupled to the needle engaging portion end 330 of the skin interface element 308.

The skin interface element 308 having a flow path 390 therein, comprised of a small diameter forward portion 392 and greater diameter rearward portion 394, forming a shoulder 393 therebetween. The forward portion 392 terminates at flow path forward end 396. There is a recessed area 398 provided between flow path forward end 396 and forward end 330. The rearward portion 394 terminates at flow path rearward end 400. The rearward portion 394 has a generally cylindrical inner surface 402.

The connector engaging portion 332 having first two opposite faces 404. A connector locking protrusion 412 is positioned on the face 404. The face 404 defines a forwardly facing edge 405.

The second two opposite faces 414 are each forming a rotational recess 416. The rotational recess 416 may be formed between a forwardly disposed connecting flange 418, which is connecting between opposed faces 404 and between a rearwardly disposed connecting wall 420, which is connecting between opposed faces 404 and terminates at rearward end 328.

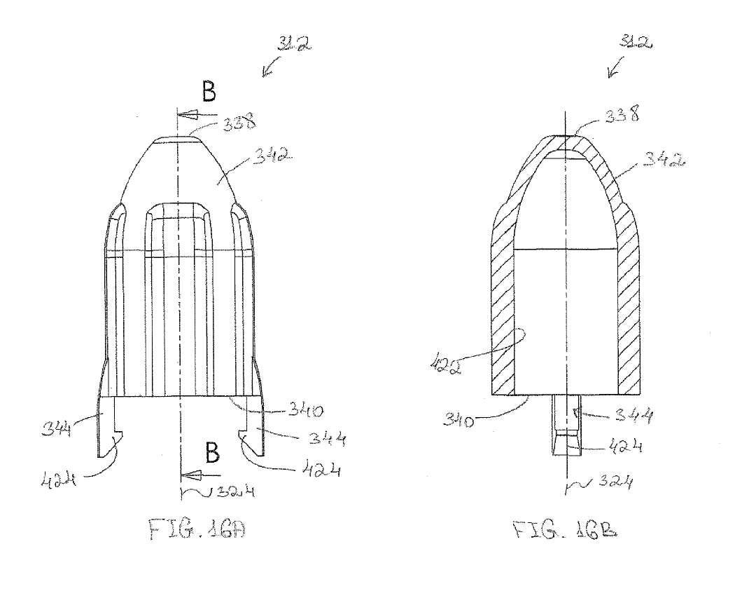

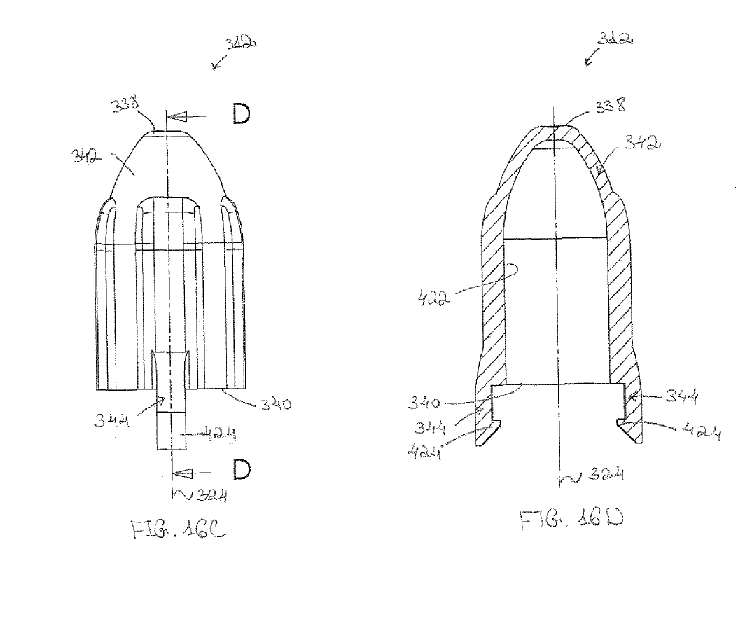

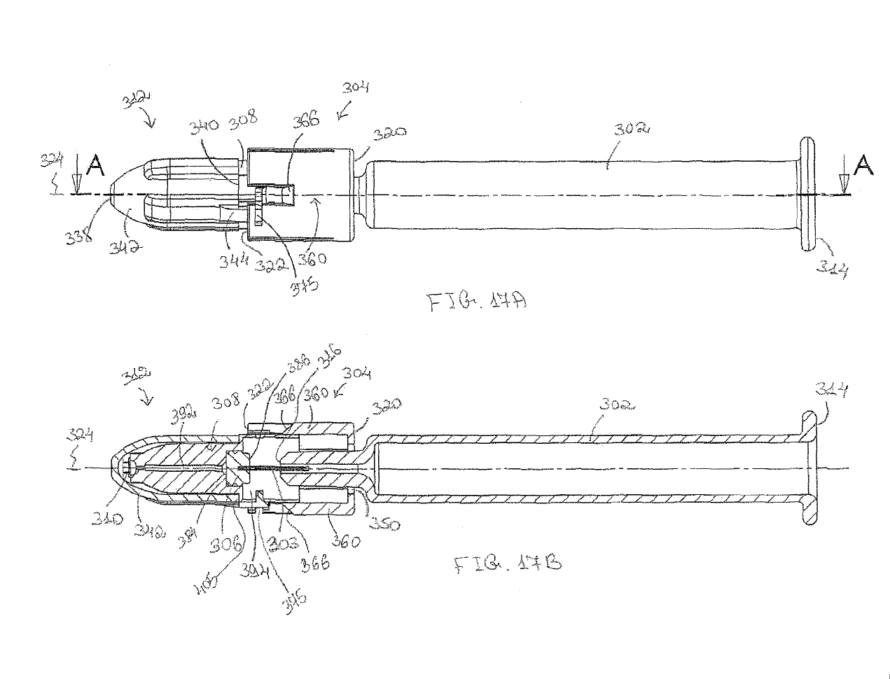

Reference is now made to FIGS. 16, 16A-16D, which illustrate the shield element 312 forming part of the medication delivery assembly 300 of FIGS. 11-12B. The shield element 312 may be an integrally formed element, preferably formed of plastic, which is symmetric about a longitudinal axis, such as axis 324 (FIGS. 11-12B).

As noted hereinabove with reference to FIGS. 11-12B, the shield 312 may be arranged along a longitudinal axis 324 and have a forwardly facing edge 338 and a rearwardly facing edge 340, which are connected by an outer surface 342. The shield 312 further has locking arms 344 extending partially rearwardly of the rearward edge 340. The shield 312 is adapted to cover the skin interface element 308.

The shield 312 further defines an inner surface 422. The locking arms 344 having an integrally formed, generally rearwardly disposed skin interface element locking protrusions 424. The locking protrusions 424 are extending internally from the outer surface of the locking arms 344.

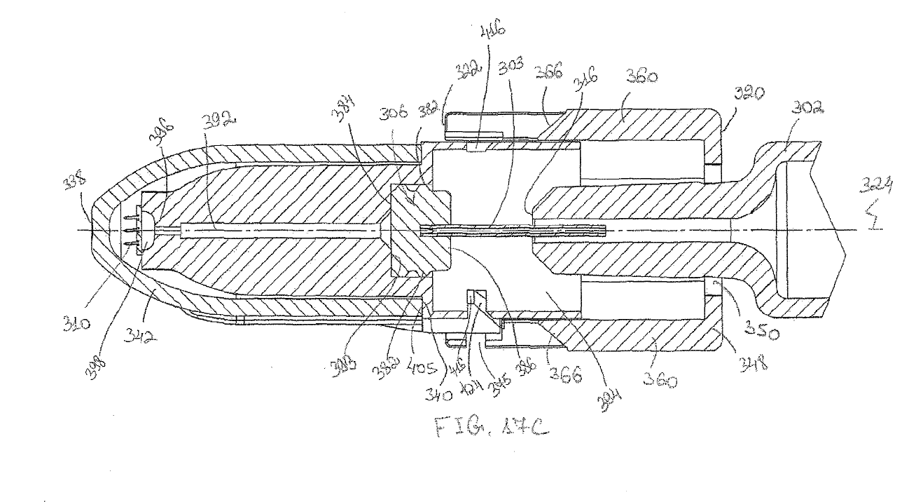

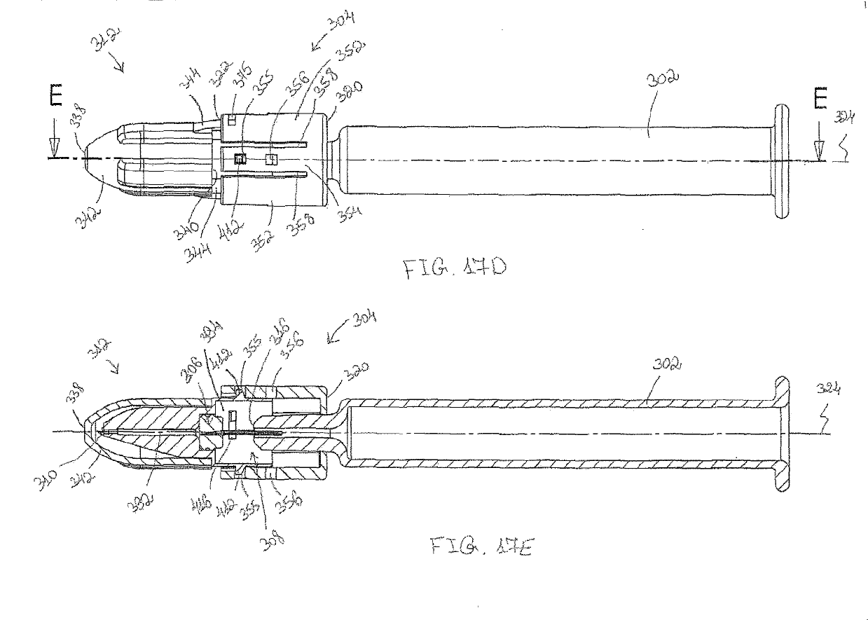

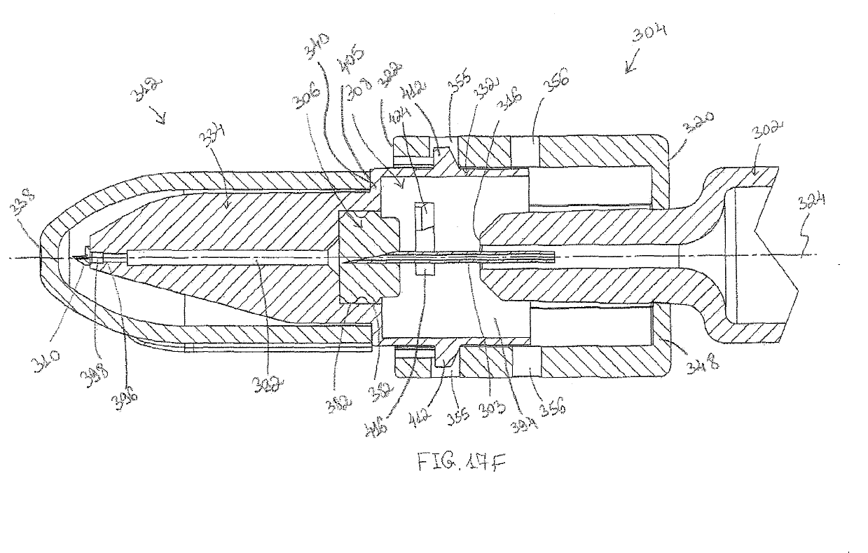

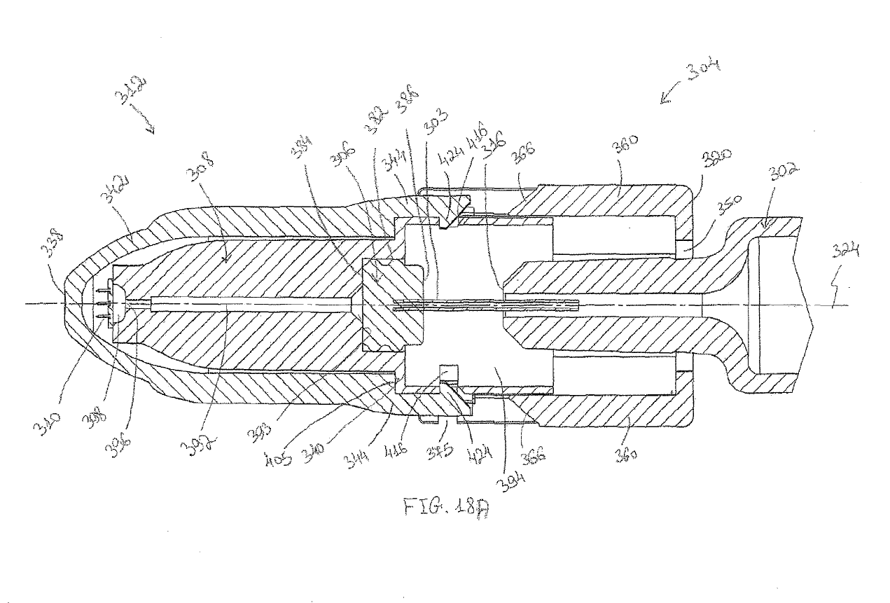

Reference is now made to FIGS. 17A-17F, which are sectional illustrations of the medication delivery assembly 300 of FIG. 11 in an inactive operative position, while in engagement with the prefilled injection device 302.

It can be seen from the above mentioned drawings showing the medication delivery assembly 300 in an inactive position that the medication delivery assembly 300 is attached to a pre-filled injection device 302. The prefilled injection device 302 may be attached to the connector 304 of medication delivery assembly 300 by means of a stopping rim 317, positioned on the luer portion 315 of the prefilled injection device 302. The luer portion 315 of the injection device 302 is inserted through the aperture 350 of the connector 304. The rim of the aperture 350 is preferably segmented and slightly undersized for the lip of the stopping rim 317, so that the rim of the aperture 350 momentarily flexes outwards as the luer portion 315 is inserted through the aperture 350 of the connector 304 and snaps into place behind the stopping rim 317.

The connector 304 and the injection device 302 then become permanently attached such that they cannot be readily released from each other in a non-destructive manner. It is appreciated that the injection device 302 may be integrally formed with the connector 304, for example by means of injection molding.

It can also be seen from the above mentioned drawings that the skin interface element 308 at the inactive position may be engaged with the connector 304 in a first lockable manner. A connector locking protrusion 412 on the skin interface element 308 is engaged in a lockable manner within the forward skin interface element holding recess 355.

The septum 306 may be located within the skin interface element 308 flow path 390 and securely held within by means of annular rings 382 that are frictionally held against the cylindrical inner surface 402. The annular rings 382 are also providing a seal by preventing the fluid from the prefilled injection device 302 that is flowing through the flow path 390 from flowing around the septum 306. The septum is spaced from the luer portion 315 of the prefilled injection device 302.

The sharp end of the needle 303 of the prefilled injection device 302 extends into the septum 306 at the inactive position and does not penetrate the septum therethrough. The sharp end of the needle 303 is not exposed in this position, thus fluid flow is not permitted.

The microneedle chip 310 may be permanently attached to the forward end 330 of the skin interface element 308.

The shield 312 may be attached to the skin interface element 308 at the inactive position. The rearwardly facing edge 340 of the shield 112 may be disposed adjacent to the forwardly facing edge 405 of the skin interface element 308.

It can further be seen that the skin interface element locking protrusions 424 of the shield 312 are out of engagement with the rotational recess 416 of the skin interface element 308 and the rotational recess 416 are in turn out of engagement with the skin interface element engaging recess 375 of the connector 304. The locking arms 344 of the shield 312 are held between the faces 414 of the skin interface element 308 and between the inner circumferences 326 of the connector 304.

It is appreciated that the medication delivery assembly 300 in the state shown in FIGS. 17A-17F prevents inadvertent microneedle puncturing and disposal of medication by means of shielding the microneedle chip 310 and plugging the needle 303 of the prefilled injection device 302.

It is further seen from the abovementioned drawings that the shield 312 cannot be axially displaced from the inactive position shown in FIGS. 17A-17F, since the skin interface element locking protrusions 424 of the shield 312 are out of engagement with the rotational recess 416 of the skin interface element 308 and the rotational recesses 416 are in turn out of engagement with the skin interface element engaging recess 375 of the connector 304. The shield 312 can be only rotationally displaced from the inactive position.

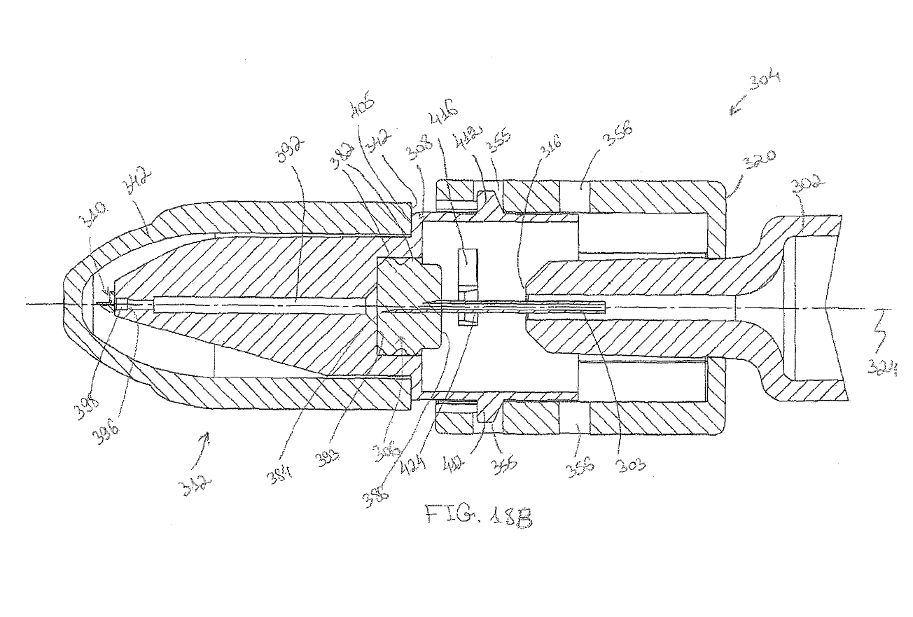

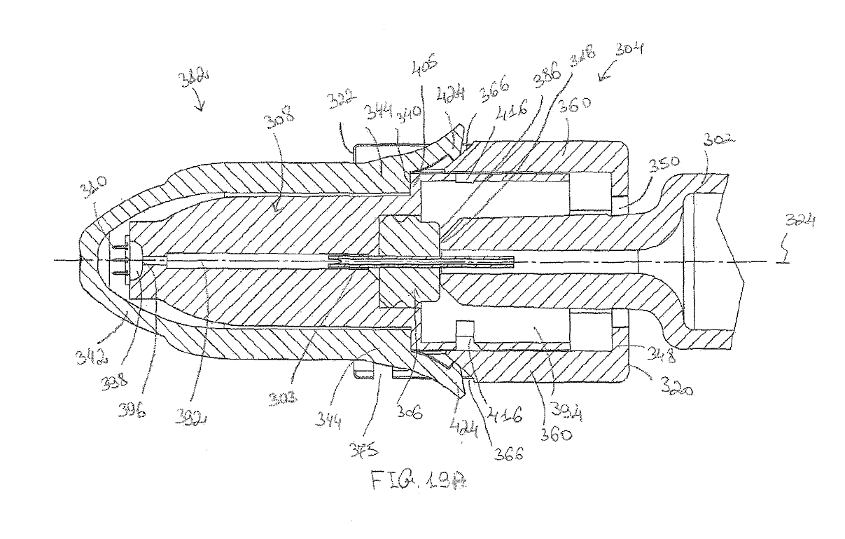

Reference is now made to FIGS. 18A and 18B, which are simplified partial enlargement of sectional illustration of the medication delivery assembly 300 of FIG. 11 in a first active operative position, while in engagement with the prefilled injection device 302.

It can be seen from the above mentioned drawings showing the medication delivery assembly 300 in a first active position that the medication delivery assembly 300 may be attached to a pre-filled injection device 302. The prefilled injection device 302 may be attached to the connector 304 of medication delivery assembly 300 by means of a stopping rim 317, positioned on the luer portion 315 of the prefilled injection device 302. The luer portion 315 of the injection device 302 is inserted through the aperture 350 of the connector 304. The rim of the aperture 350 is preferably segmented and slightly undersized for the lip of the stopping rim 317, so that the rim of the aperture 350 momentarily flexes outwards as the luer portion 315 is inserted through the aperture 350 of the connector 304 and snaps into place behind the stopping rim 317.

The connector 304 and the injection device 302 then become permanently attached such that they cannot be readily released from each other in a non-destructive manner. It is appreciated that the injection device 302 may be integrally formed with the connector 304, for example by means of injection molding.

It can also be seen from the above mentioned drawings that the skin interface element 308 at the first active position is engaged with the connector 304 in a first lockable manner. The shield 312 is rotationally displaced from the deactivated position shown in FIGS. 17A-17F. This displacement urges the skin interface element locking protrusions 424 into engagement with the rotational recess 416 of the skin interface element 308. Simultaneously, the rotational recess 416 of the skin interface element 308 are urged into engagement with the skin interface element engaging recess 375 of the connector 304, causing the skin interface element locking protrusions 424 to lockably engage the rotational recess 416 of the skin interface element 308 and further causing the rotational recess 416 of the skin interface element 308 to lockably engage the skin interface element engaging recess 375 of the connector 304.

Following the abovementioned engagement, the connector locking protrusions 412 on the skin interface element 308 are engaged in a lockable manner within the forward skin interface element holding recess 355.

The septum 306 may be located within the skin interface element 308 flow path 390 and securely held within by means of annular rings 382 that are frictionally held against the cylindrical inner surface 402. The annular rings 382 are also providing a seal by preventing the fluid from the prefilled injection device 302 that is flowing through the flow path 390 from flowing around the septum 306. The septum is spaced from the luer portion 315 of the prefilled injection device 302.

The sharp end of the needle 303 of the prefilled injection device 302 extends into the septum 306 at the first activated position and does not pierce the septum 306 therethrough. The sharp end of the needle 303 is not exposed in this position, thus fluid flow is not permitted.

The microneedle chip 310 may be permanently attached to the forward end 330 of the skin interface element 308.

The shield 312 may be attached to the skin interface element 308 at the first active position, as it is described above. The rearwardly facing edge 340 of the shield 312 is disposed adjacent to the forwardly facing edge 405 of the skin interface element 308.

It is appreciated that the medication delivery assembly 300 in the state shown in FIGS. 18A and 18B prevents inadvertent microneedle puncturing and disposal of medication by means of shielding the microneedle chip 310 and plugging the needle 303 of the prefilled injection device 302.

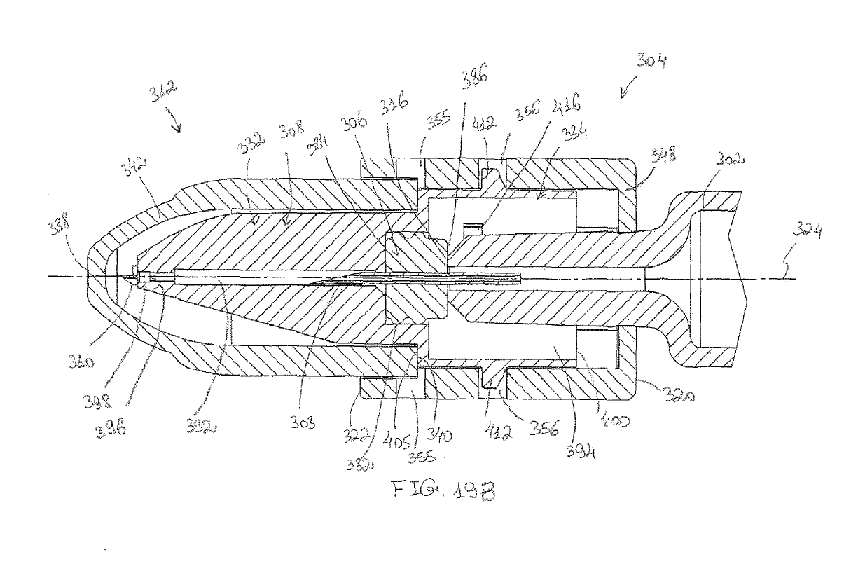

Reference is now made to FIGS. 19A and 19B, which are simplified partial enlargement of sectional illustration of the medication delivery assembly 300 of FIG. 11 in a second active operative position, while in engagement with the prefilled injection device 302.

It can be seen from the above mentioned drawings showing the medication delivery assembly 300 in the second active position that the medication delivery assembly 300 may be attached to a pre-filled injection device 302. The prefilled injection device 302 may be attached to the connector 304 of medication delivery assembly 300 by means of a stopping rim 317, positioned on the luer portion 315 of the prefilled injection device 302. The luer portion 315 of the injection device 302 is inserted through the aperture 350 of the connector 304. The rim of the aperture 350 is preferably segmented and slightly undersized for the lip of the stopping rim 317, so that the rim of the aperture 350 momentarily flexes outwards as the luer portion 315 is inserted through the aperture 350 of the connector 304 and snaps into place behind the stopping rim 317.

The connector 304 and the injection device 302 then become permanently attached such that they cannot be readily released from each other in a non-destructive manner. It is appreciated that the injection device 302 may be integrally formed with the connector 304, for example by means of injection molding.

It can also be seen from the above mentioned drawings that the skin interface element 308 at the second active position is engaged with the connector 304 in a second lockable manner. Following rearward displacement of the shield 312, in order to activate the medication delivery assembly 300, a connector locking protrusion 412 on the skin interface element 308 is displaced and is now engaged in a lockable manner within the rearward skin interface element holding recess 356.

The septum 306 may be located within the skin interface element 308 flow path 390 and securely held within by means of annular rings 382 that are frictionally held against the cylindrical inner surface 402. The annular rings 382 are also providing a seal by preventing the fluid from the prefilled injection device 302 that is flowing through the flow path 390 from flowing around the septum 306.

The sharp end of the needle 303 of the prefilled injection device 302 extends throughout the septum 306 at the second active position the septum rearward end 384 is disposed adjacent the forward end 316 of the prefilled injection device 302. The sharp end of the needle 303 is exposed into the forward portion 392 of the flow path 390 of the skin interface element 308 in the second active position, thus fluid flow is permitted from the prefilled injection device 302 via the needle 303, further via the forward portion 392 of the flow path 390 of the skin interface element 308 and through the microneedle array arranged on the microneedle chip 310.

In accordance to a preferred embodiment of the invention, the microneedle chip 310 may be formed of at least one hollow penetrating element, which is implemented as at least one hollow microneedle integrally formed with an underlying substrate.

The microneedle chip 310 may be preferably formed of two hollow microneedles integrally formed with an underlying substrate or may be alternatively formed of a linear array of at least three hollow microneedles integrally formed with an underlying substrate. Each microneedle within the microneedle chip 310 may be preferably formed primarily from silicon.

It may be appreciated that in a particular embodiment of the invention, each hollow microneedle is formed with at least one upright surface standing upright relative to a surface of said underlying substrate, an inclined surface intersecting said at least one upright surface and a fluid flow bore intersecting said inclined surface.

In accordance to an embodiment of the invention, each hollow microneedle is preferably less than 1 mm of height.

Each hollow microneedle is located adjacent to an edge of said underlying substrate in such a manner that the microneedle having a height, and being less than twice its own height away from the edge.

It is further appreciated that the microneedle chip 310 may be constructed as it is previously disclosed in U.S. Pat. Nos. 7,648,484 and 6,533,949, assigned to Nanopass Technologies.

The microneedle chip 310 may be permanently attached to the forward end 330 of the skin interface element 308.

The shield 312 is disposed over the skin interface element 308 at the second active position; however it is no longer attached to the skin interface element 308. The rearwardly facing edge 340 of the shield 312 is still disposed adjacent to the forwardly facing edge 405 of the skin interface element 308.

It can further be seen that the skin interface element locking protrusions 424 of the shield 312 are no longer engaged within the rotational recess 416. Due to manual rearward displacement of the shield 312, the skin interface element 308 is displaced rearwardly as well, the connector locking protrusion 412 of the skin interface element 308 is moving out of engagement with the forward skin interface element holding recess 355 of the connector 304 and becomes instead locked within the rearward holding recess 356 of the connector 304. The locking of the connector locking protrusion 412 with the holding recess 356 is made permanent due to the structure of the locking protrusions 412, which have one straight end and one sloped end, such that the connector 304 and the skin interface element 308 cannot be unlocked unless sufficient force is exerted to overcome this locking relation that is not readily achieved manually.

Simultaneously, due to the rearward movement of the skin interface element 308, the locking arms 344 of the shield 312 are deflected outwardly and are sliding generally rearwardly over the sloped end 366 of the raised wail 360 of the connector 304.

The rearward end 328 of the skin interface element 308 may be positioned adjacent the connecting wall 348 of the connector 304 at the second active position.

It is appreciated that the medication delivery assembly 300 in the state shown in FIGS. 19A and 19B is a transitional stage of activation, which still doesn't allow inadvertent microneedle puncturing, however the shield 312 is released from lockable engagement at this stage and is ready to be removed from the medication delivery assembly 300.

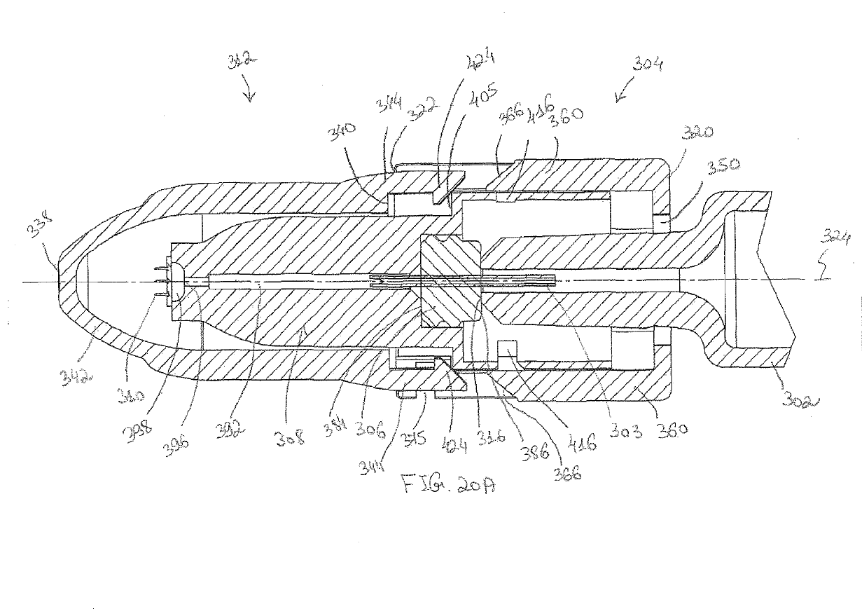

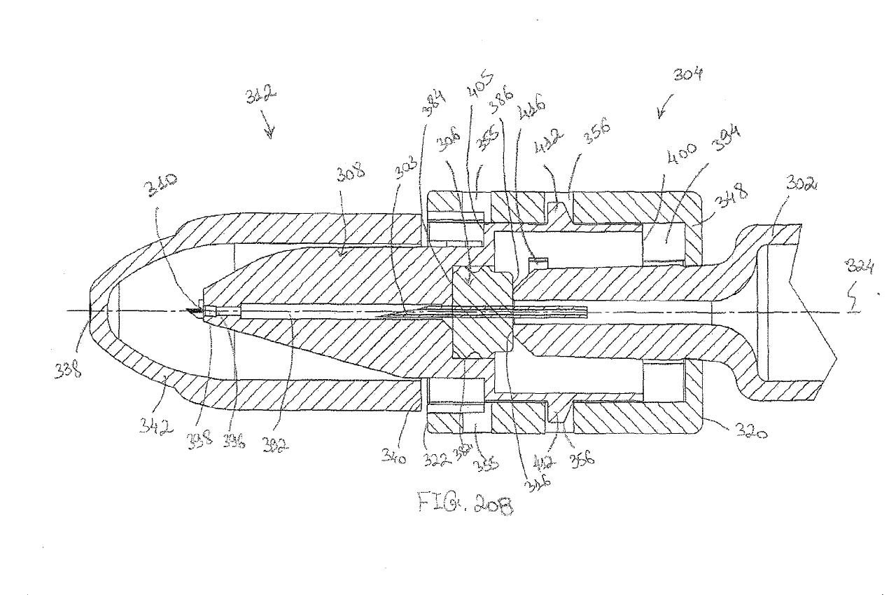

Reference is now made to FIGS. 20A and 20B, which are simplified partial enlargement of sectional illustration of the medication delivery assembly 300 of FIG. 11 in an open operative position, while in engagement with the prefilled injection device 302.

It can be seen from the above mentioned drawings showing the medication delivery assembly 300 in an open position that the medication delivery assembly 300 may be attached to a pre-filled injection device 302. The prefilled injection device 302 may be attached to the connector 304 of medication delivery assembly 300 by means of a stopping rim 317, positioned on the luer portion 315 of the prefilled injection device 302. The luer portion 315 of the injection device 302 is inserted through the aperture 350 of the connector 304. The rim of the aperture 350 is preferably segmented and slightly undersized for the lip of the stopping rim 317, so that the rim of the aperture 350 momentarily flexes outwards as the luer portion 315 is inserted through the aperture 350 of the connector 304 and snaps into place behind the stopping rim 317.

The connector 304 and the injection device 302 then become permanently attached such that they cannot be readily released from each other in a non-destructive manner. It is appreciated that the injection device 302 may be integrally formed with the connector 304, for example by means of injection molding.

It can also be seen from the above mentioned drawings that the skin interface element 308 at the open stage is engaged with the connector 304 in a second lockable manner.

As previously shown in FIGS. 19A and 19B, following rearward displacement of the shield 312, in order to activate the medication delivery assembly 300, a connector locking protrusion 412 on the skin interface element 308 is displaced and engaged in a lockable manner within the rearward skin interface element holding recess 356.

In an open operative position shown in FIGS. 20A and 20B, the shield 312 is still disposed over the skin interface element 308.

The shield 312 is released from rearward displacement and consequentially the locking arms 344 of the shield 312 are not deflected anymore, thus the locking arms 344 return to their normal position while sliding generally forwardly along the sloped end 366 of the raised wall 360 of the connector 304. The locking arms 344 of the shield 312 in the open operative position are disposed between the two lateral portions 352 of the connector 304, however the arms 344 are not held at this position, as it was shown in FIGS. 18A and 18B in the first active position, since the skin interface element locking protrusions 424 of the shield 312 are out of engagement with the shield locking portion 416 of the skin interface element 308 following the second activation stage, as described with reference to FIGS. 19A and 19B.

While referring specifically to FIGS. 20A and 20B, the rearwardly facing edge 340 of the shield 312 is spaced from the forwardly facing edge 405 of the skin interface element 308 as the shield 312 is moving forwardly.

The skin interface element 308 at the open operative stage is displaced rearwardly, the connector locking protrusion 412 of the skin interface element 308 are engaged with the rearward skin interface element holding recess 356 of the connector 304.

The septum 306 may be located within the skin interface element 308 flow path 390 and securely held within by means of annular rings 382 that are frictionally held against the cylindrical inner surface 402. The annular rings 382 are also providing a seal by preventing the fluid from the prefilled injection device 302 that is flowing through the flow path 390 from flowing around the septum 306.

The sharp end of the needle 303 of the prefilled injection device 302 extends throughout the septum 306 at the open operative position. The septum rearward end 384 may be disposed adjacent the forward end 316 of the prefilled injection device 302. The sharp end of the needle 303 is exposed into the forward portion 392 of the flow path 390 of the skin interface element 308 in the open operative position, thus fluid flow is permitted from the prefilled injection device 302 via the needle 303, further via the forward portion 392 of the flow path 390 of the skin interface element 308 and through the microneedle array arranged on the microneedle chip 310. In accordance to a preferred embodiment of the invention, the microneedle chip 310 may be formed of at least one hollow penetrating element, which is implemented as at least one hollow microneedle integrally formed with an underlying substrate.

The microneedle chip 310 may be preferably formed of two hollow microneedles integrally formed with an underlying substrate or may be alternatively formed of a linear array of at least three hollow microneedles integrally formed with an underlying substrate.

Each microneedle within the microneedle chip 310 may be preferably formed primarily from silicon.

It may be appreciated that in a particular embodiment of the invention, each hollow microneedle is formed with at least one upright surface standing upright relative to a surface of said underlying substrate, an inclined surface intersecting said at least one upright surface and a fluid flow bore intersecting said inclined surface.

In accordance to an embodiment of the invention, each hollow microneedle is preferably less than 1 mm of height.

Each hollow microneedle is located adjacent to an edge of said underlying substrate in such a manner that the microneedle having a height, and being less than twice its own height away from the edge.

It is further appreciated that the microneedle chip 310 may be constructed as it is previously disclosed in U.S. Pat. Nos. 7,648,484 and 6,533,949, assigned to Nanopass Technologies.

The microneedle chip 310 may be permanently attached to the forward end 330 of the skin interface element 308.

The rearward end 328 of the skin interface element 308 is fixedly positioned adjacent the connecting wall 348 of the connector 304 at the open operative position.

It is appreciated that the medication delivery assembly 300 in the state shown in FIGS. 20A and 20B is a transitional stage of releasing the shield 312, which still doesn't allow inadvertent microneedle puncturing.

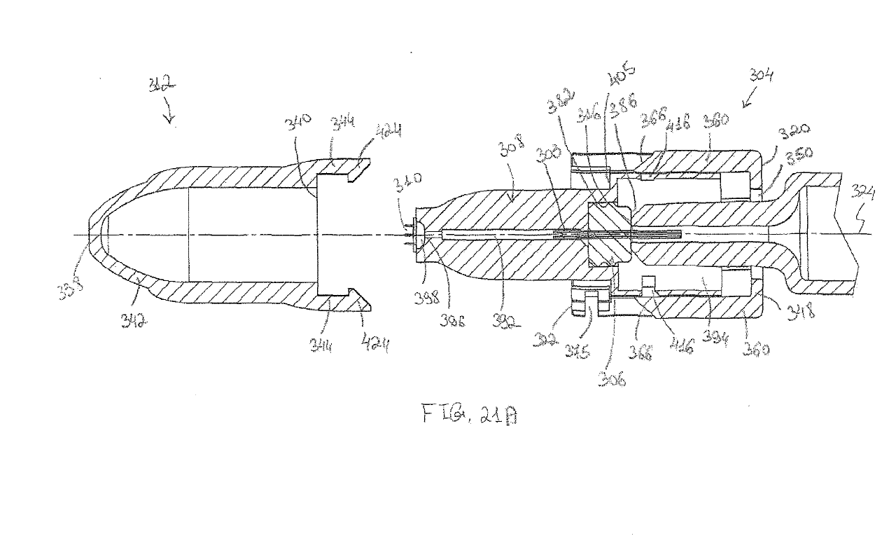

Reference is now made to FIGS. 21A and 21B, which are simplified partial enlargement of sectional illustration of the medication delivery assembly 300 of FIG. 11 in an injection position, while in engagement with the prefilled injection device 302.

It can be seen from the above mentioned drawings showing the medication delivery assembly 300 in an injection position that the medication delivery assembly 300 may be attached to a pre-filled injection device 302. The prefilled injection device 302 may be attached to the connector 304 of medication delivery assembly 300 by means of a stopping rim 317, positioned on the luer portion 315 of the prefilled injection device 302. The luer portion 315 of the injection device 302 is inserted through the aperture 350 of the connector 304. The rim of the aperture 350 is preferably segmented and slightly undersized for the lip of the stopping rim 317, so that the rim of the aperture 350 momentarily flexes outwards as the luer portion 315 is inserted through the aperture 350 of the connector 304 and snaps into place behind the stopping rim 317.

The connector 304 and the injection device 302 then become permanently attached such that they cannot be readily released from each other in a non-destructive manner. It is appreciated that the injection device 302 may be integrally formed with the connector 304, for example by means of injection molding.

It can also be seen from the above mentioned drawings that the skin interface element 308 at the injection stage may be fixedly engaged with the connector 304 in a second lockable manner.

As previously shown in FIGS. 19A and 19B, following rearward displacement of the shield 312 in order to activate the medication delivery assembly 300, a connector locking protrusion 412 on the skin interface element 308 is displaced and engaged in a lockable manner within the rearward skin interface element holding recess 356.

In the injection position shown in FIGS. 21A and 21B, the shield 312 is removed completely from the skin interface element 308.

The skin interface element 308 at the injection position is disposed rearwardly, the connector locking protrusion 412 of the skin interface element 308 are engaged with the rearward skin interface element holding recess 356 of the connector 304.

The septum 306 may be located within the skin interface element 308 flow path 390 and securely held within by means of annular rings 382 that are frictionally held against the cylindrical inner surface 402. The annular rings 382 are also providing a seal by preventing the fluid from the prefilled injection device 302 that is flowing through the flow path 390 from flowing around the septum 306.

The sharp end of the needle 303 of the prefilled injection device 302 extends throughout the septum 306 at the injection position. The septum rearward end 384 is disposed adjacent the forward end 316 of the prefilled injection device 302. The sharp end of the needle 303 is exposed into the forward portion 392 of the flow path 390 of the skin interface element 308 in the open operative position, thus fluid flow is permitted from the prefilled injection device 302 via the needle 303, further via the forward portion 392 of the flow path 390 of the skin interface element 308 and through the micro needle array arranged on the micro needle chip 310.

In accordance to a preferred embodiment of the invention, the microneedle chip 310 may be formed of at least one hollow penetrating element, which is implemented as at least one hollow microneedle integrally formed with an underlying substrate.

The microneedle chip 310 may be preferably formed of two hollow microneedles integrally formed with an underlying substrate or may be alternatively formed of a linear array of at least three hollow microneedles integrally formed with an underlying substrate.

Each microneedle within the microneedle chip 310 may be preferably formed primarily from silicon.

It may be appreciated that in a particular embodiment of the invention, each hollow microneedle is formed with at least one upright surface standing upright relative to a surface of said underlying substrate, an inclined surface intersecting said at least one upright surface and a fluid flow bore intersecting said inclined surface.

In accordance to an embodiment of the invention, each hollow microneedle is preferably less than 1 mm of height.

Each hollow microneedle is located adjacent to an edge of said underlying substrate in such a manner that the microneedle having a height, and being less than twice its own height away from the edge.

It is further appreciated that the microneedle chip 310 may be constructed as it is previously disclosed in U.S. Pat. Nos. 7,648,484 and 6,533,949, assigned to Nanopass Technologies.

The microneedle chip 310 may be permanently attached to the forward end 330 of the skin interface element 308.

The rearward end 328 of the skin interface element 308 is fixedly positioned adjacent the connecting wall 348 of the connector 304 at the injection position.

It is appreciated that the medication delivery assembly 300 in the state shown in FIGS. 21A and 21B is an injection stage while the shield 312 is completely removed and injection of fluid from the prefilled injection device 302 throughout the medication delivery assembly 300 and through the microneedles 310 is permitted.

It is appreciated that the medication delivery assembly 300 as shown in FIG. 11 requires two stages of activation in order to allow injection of fluid from the prefilled injection device 302. First activation stage is performed by means of rotational displacement of the shield 312 relative the connector 304 as shown in FIGS. 18A and 18B and second activation stage is performed by means of axial displacement of the shield 312 relative the connector 304 as shown in FIGS. 19A and 19B.

It is further appreciated that following the injection, the shield 312 may be placed back onto the skin interface element 308 as it is shown on FIGS. 20A and 2013. At this position, the shield 312 covers the microneedle chip 310, which provides for another safety functionality by preventing inadvertent needle puncturing at the discarding stage.

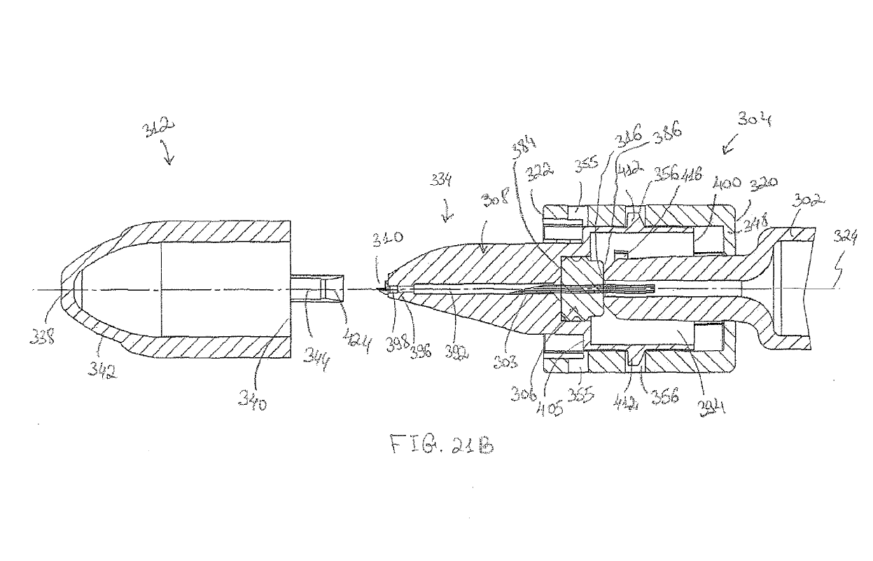

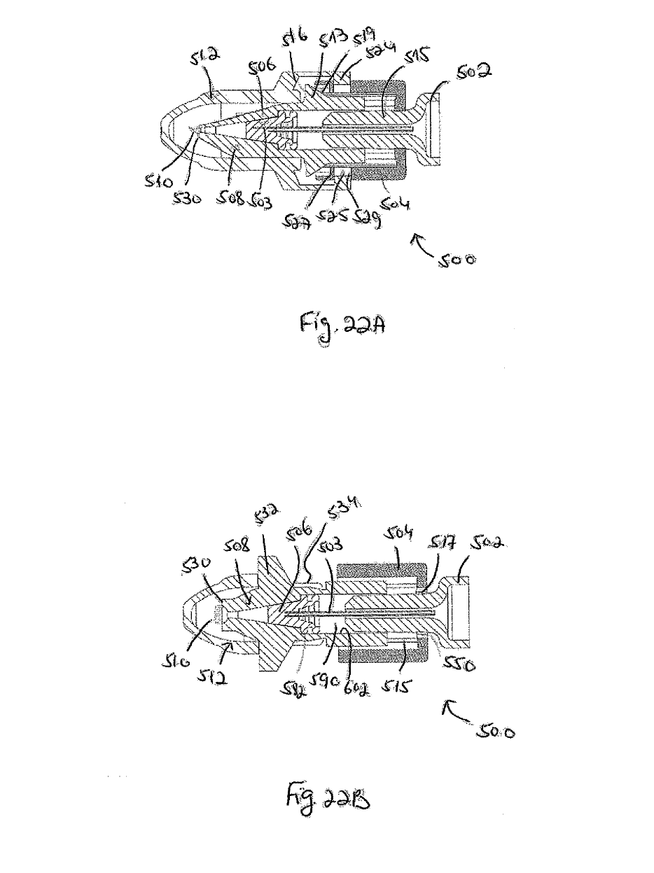

Reference is now made to FIGS. 22A and 22B, which are simplified enlargement orthogonal cross sectional view illustrations of a medication delivery assembly in an inactive operative position constructed and operative in accordance with another preferred embodiment of the invention.

FIGS. 22A and 22B are respective illustrations to FIGS. 7C and 7F, showing another preferred embodiment of the invention.

FIGS. 22A and 22B show a medication delivery assembly 500 in an inactive position that may be attached to a pre-filled injection device 502. The prefilled injection device 502 may be attached to a connector 504 of medication delivery assembly 500 by means of a stopping rim 517, positioned on a luer portion 515 of the prefilled injection device 502.

The luer portion 515 of the injection device 502 is inserted through an aperture 550 of the connector 504. The rim of the aperture 550 is preferably segmented and slightly undersized for the lip of the stopping rim 517, so that the rim of the aperture 550 momentarily flexes outwards as the luer portion 515 is inserted through the aperture 550 of the connector 504 and snaps into place behind the stopping rim 517.

The connector 504 and the injection device 502 then become permanently attached such that they cannot be readily released from each other in a non-destructive manner. It is appreciated that the injection device 502 may be integrally formed with the connector 504, for example by means of injection molding.

It can also be seen from the above mentioned drawings that a skin interface element 508 at the inactive position may be movably disposed at least partially within the connector 504. Connector locking protrusions 513 on the skin interface element 508, having one straight face 516 and one sloped end 519, are not engaged with the connector 504 at the inactive position.

A septum 506 may be located within the skin interface element 508 flow path 590 and may be securely held within by means of annular rings 582 that are frictionally held against the cylindrical inner surface 602. The annular rings 582 may also provide a seal by preventing the fluid from the prefilled injection device 502 that is flowing through the flow path 590 from flowing around the septum 506. The septum is spaced from the lure portion 515 of the prefilled injection device 502.

The sharp end of a needle 503 of the pre-filled injection device 502 is extending into the septum 506 without piercing the septum therethrough at the inactive position. The sharp end of the needle 503 is not exposed in this position, thus fluid flow is not permitted.

A microneedle chip 510 is preferably permanently attached to a forward end 530 of the skin interface element 508.

The embodiment of FIGS. 22A and 22B differs from the previously described embodiments primarily in that the transition from the inactive to the active states occurs through motion of skin interface element 508 alone, without motion of its shield 512. In the non-limiting example illustrated here, shield 512 is initially fixedly attached to the connector 504 while the skin interface element is in the inactive position. Connector locking arms 524 of the shield 512 are fixedly engaged within skin interface element locking recesses 525 of the connector 504 in a lockable manner, such that the connector locking arms 524 cannot be removed from the skin interface element locking recesses 525 without outward deflection of the locking arms 524. The skin interface element locking recesses 525 are defined by two opposed ends, a forward end 527 having a slightly sloped angle and a straight rearward end 529. The locking arms 524 of the shield 512 are supported by the rearward end 529 of the skin interface element locking protrusions 525, thus axial rearward displacement of the shield 512 is not permitted.

It can be further seen specifically in FIG. 2213 that he skin interface element 508 further has outwardly extending gripping wings 532, which are configured to protrude through recesses 534 in the shield 512 and thus provide gripping surface to allow axial displacement of the skin interface element 508 relative to the shield 512.

It is appreciated that the medication delivery assembly 500 in the state shown in FIGS. 22A and 22B is capable of preventing inadvertent microneedle puncturing and disposal of medication by means of shielding the microneedle chip 510 and plugging the needle 503 of the prefilled injection device 502.

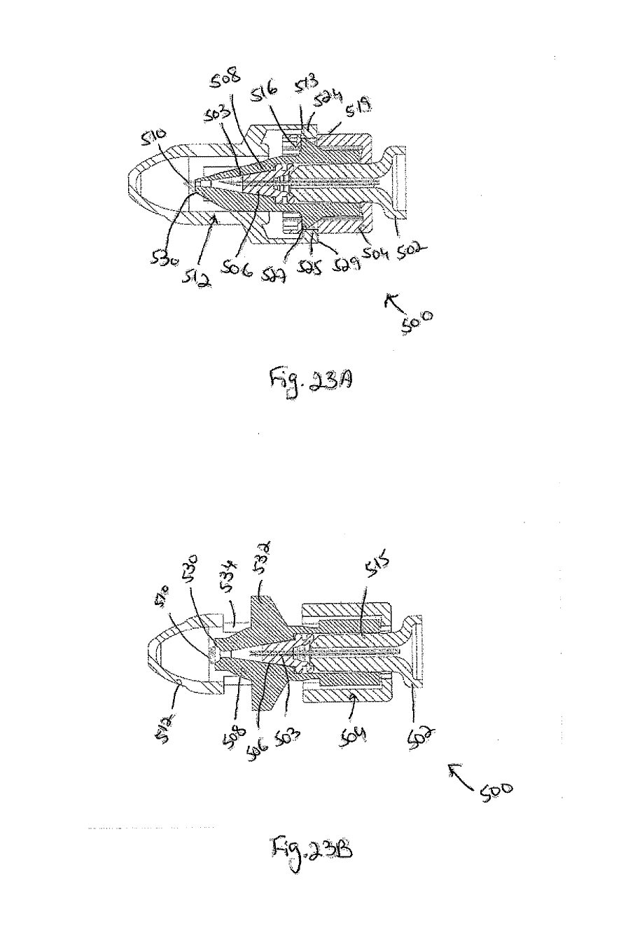

Reference is now made to FIGS. 23A and 23B, which are simplified enlargement orthogonal cross sectional view illustrations of a medication delivery assembly of FIGS. 22A and 22B.

It can be seen from the above mentioned drawings showing the medication delivery assembly 500 in an active position that the medication delivery assembly 500 may be attached to a pre-filled injection device 502. The prefilled injection device 502 may be attached to the connector 504 of medication delivery assembly 500 by means of a stopping rim 517, positioned on the luer portion 515 of the prefilled injection device 502. The luer portion 515 of the injection device 502 is inserted through the aperture 550 of the connector 504. The rim of the aperture 550 is preferably segmented and slightly undersized for the lip of the stopping rim 517, so that the rim of the aperture 550 momentarily flexes outwards as the luer portion 515 is inserted through the aperture 550 of the connector 504 and snaps into place behind the stopping rim 517.

The connector 504 and the injection device 502 then become permanently attached such that they cannot be readily released from each other in a non-destructive manner. It is appreciated that the injection device 502 may be integrally formed with the connector 504, for example by means of injection molding.

It can also be seen from the above mentioned drawings that the skin interface element 508 at the active position is engaged with the connector 504 in a lockable manner. The skin interface element 508 is axially rearwardly disposed due the manual force exerted on the gripping wings 532, which are slidable along the recesses 534 of the shield 512 in order to activate the medication delivery assembly 500, i.e., to make it ready for use. The sloped face 519 of the connector locking protrusion 513 of the skin interface element 508 slides along the inner surface of the connector 504 and the straight face 516 of the connector locking protrusions 513 on the skin interface element 508 may then snap over the forward end 527 of the skin interface element locking recesses 525 of the connector 504 and become engaged in a lockable manner within the skin interface element locking recesses 525 of the connector 504, between the forward end 527 and the rearward end 529 of the skin interface element locking recesses 525.

The rearward displacement of the skin interface element 508 and snapping behind the forward end 527 of the skin interface element locking recesses 525 is permitted due to a substantial resiliency of the material that the skin interface element 508 and/or connector 504 are made from, optionally in combination with various cut-outs or other geometrical features designed to accommodate the required momentary deflection and then return resiliently towards their original shapes.

The rearward displacement of the skin interface element 508 causes the connector locking protrusions 513 of the skin interface element 508 to be inserted into the skin interface element locking recesses 525 and thereby urges the connector locking arms 524 of the shield 512 to deflect outwardly and thus disengage from the skin interface locking recesses 525 of the connector 504 sufficiently to allow manual removal of the shield.

While in the activation position, the connector locking arms 524 of the shield 512 cannot be axially displaced since they are locked between the forward end 527 and the rearward end 529 of the skin interface element locking recesses 525.

Following the engagement of the connector locking protrusions 513 of the skin interface element 508 with the skin interface element locking recesses 525, the connector locking arms 524 of the shield 512 are thus released and can be displaced forwardly and slide along the sloped angle of the forward end 527 of the skin interface element locking recesses 525 and thus the shield 512 can be removed to uncover the microneedle chip 510 for injection of medication. Parenthetically, it should be noted that the term “release” as used herein throughout the description and claims refers to a transition from a state that cannot readily be removed or separated manually to a state that can readily be removed or separated manually, but does not preclude there being a remnant retention force which must be manually overcome in order to actually remove the shield. For example, in the present embodiment, removal of shield 512 requires application of forward force in order to slightly flex locking arms 524 further outwards as the connector locking protrusions 513 ride over the outwardly sloped external bevel angle of the forward end 527 of the skin interface element locking recesses 525.

It may be appreciated that a single rearward axial movement of the skin interface element 508 causes both activation of the medication delivery device 500 by engaging the connector locking protrusions 513 of the skin interface element 508 with the skin interface element locking protrusions 525 of the connector 504 and release of the connector locking arms 524 of the shield 512 from the connector 504.

The septum 506 may be located within the skin interface element 508 flow path 590 and may be securely held within by means of annular rings 582 that are frictionally held against the cylindrical inner surface 602. The annular rings 582 are also providing a seal by preventing the fluid from the prefilled injection device 502 that is flowing through the flow path 590 from flowing around the septum 506.

The sharp end of the needle 503 of the prefilled injection device 502 may extend throughout the septum 506 at the active position. The septum rearward end 584 is disposed adjacent the forward end 516 of the prefilled injection device 502. The forward end 516 of the pre-filled injection device 502 may supports the septum 506 and thus prevent rearward movement of the septum 506 due to back pressure of the medication. The sharp end of the needle 503 may be exposed into the forward portion 592 of the flow path 590 of the skin interface element 508 in the active position, thus fluid flow may be permitted from the prefilled injection device 502 via the needle 503, further via the forward portion 592 of the flow path 590 of the skin interface element 508 and through the microneedle array arranged on the microneedle chip 510.

In accordance to a preferred embodiment of the invention, the microneedle chip 510 may be formed of at least one hollow penetrating element, which is implemented as at least one hollow microneedle integrally formed with an underlying substrate.

The microneedle chip 510 may be preferably formed of two hollow microneedles integrally formed with an underlying substrate or may be alternatively formed of a linear array of at least three hollow microneedles integrally formed with an underlying substrate.

Each microneedle within the microneedle chip 510 may be preferably formed primarily from silicon.

It may be appreciated that in a particular embodiment of the invention, each hollow microneedle is formed with at least one upright surface standing upright relative to a surface of said underlying substrate, an inclined surface intersecting said at least one upright surface and a fluid flow bore intersecting said inclined surface.

In accordance to an embodiment of the invention, each hollow microneedle is preferably less than 1 mm of height.

Each hollow microneedle is located adjacent to an edge of said underlying substrate in such a manner that the microneedle having a height, and being less than twice its own height away from the edge.

It is further appreciated that the microneedle chip 110 may be constructed as it is previously disclosed in U.S. Pat. Nos. 7,648,484 and 6,533,949, assigned to Nanopass Technologies.

The microneedle chip 510 may be permanently attached to the forward end 530 of the skin interface element 508.

In active position, the shield 512 may be disposed over the skin interface element 508, however it is no longer attached to the skin interface element 508 rather it can be readily removed by sliding the shield 512 forwardly along the sloped angle of the forward end 527 of the skin interface element locking recesses 525.

Due to the manual rearward displacement of the skin interface element 508, as described in detail hereinabove, the connector locking protrusions 513 of the skin interface element 508 are enabled to move into engagement with the skin interface element locking recesses 525 of the connector 504. The locking of the connector locking protrusions 513 with the skin interface element locking recesses 525 is made permanent, such that the connector 504 and the skin interface element 508 cannot be unlocked unless sufficient force is exerted to overcome this locking relation that is not readily achieved manually.

It is appreciated that the medication delivery assembly 500 in the state shown in FIGS. 23A and 23B is a transitional stage of activation, which still doesn't allow inadvertent microneedle puncturing, however the shield 512 is released from lockable engagement at this stage and is ready to be removed from the medication delivery assembly 500 and the hollow needle 503 penetrates entirely through the septum 506.

It will be appreciated by persons skilled in the art that the present invention is not limited by what has been particularly shown and described hereinabove. Rather the scope of the present invention includes both combinations and sub combinations of various features described hereinabove as well as variations and modifications thereof which are not in the prior art.

What is claimed is:

1. A drug delivery assembly for use in association with an outlet port of an injection device, the assembly comprising:
(a) a connector for association with the outlet port of the injection device;
(b) a skin interface element including a fluid flow channel in fluid connection with at least one hollow penetrating element deployed for penetrating into a biological barrier; and
(c) a shield deployed to prevent inadvertent contact with said hollow penetrating element prior to use, said shield being retained in engagement with at least one of said connector and said skin interface element,
wherein said skin interface element is mechanically engaged with said connector so as to be displaceable relative to said connector between an inactive position in which said fluid flow channel is isolated from the outlet port of the injection device and an active position in which said fluid flow channel is in fluid connection with the outlet port of the injection device,
and wherein motion of said skin interface element relative to said connector from said inactive position to said active position is effective to disengage retention of said shield.

2. The assembly of claim 1, wherein said at least one hollow penetrating element is implemented as at least one hollow microneedle integrally formed with an underlying substrate.

3. The assembly of claim 1, wherein said at least one hollow penetrating element is implemented as at least two hollow microneedles integrally formed with an underlying substrate.

4. The assembly of claim 1, wherein said at least one hollow penetrating element is implemented as a linear array of at least three hollow microneedles integrally formed with an underlying substrate.

5. The assembly of claim 2, wherein each microneedle is formed primarily from silicon.

6. The assembly of claim 3, wherein each microneedle is formed primarily from silicon.

7. The assembly of claim, wherein each microneedle is formed primarily from silicon.

8. The assembly of claim 2, wherein each hollow microneedle is formed with at least one upright surface standing upright relative to a surface of said underlying substrate, an inclined surface intersecting said at least one upright surface and a fluid flow bore intersecting said inclined surface.

9. The assembly of claim 3, wherein each hollow microneedle is formed with at least one upright surface standing upright relative to a surface of said underlying substrate, an inclined surface intersecting said at least one upright surface and a fluid flow bore intersecting said inclined surface.

10. The assembly of, wherein each hollow microneedle is formed with at least one upright surface standing upright relative to a surface of said underlying substrate, an inclined surface intersecting said at least one upright surface and a fluid flow bore intersecting said inclined surface.

11. The assembly of claim, wherein each hollow microneedle is located adjacent to an edge of said underlying substrate.

12. The assembly of claim 3, wherein each hollow microneedle is located adjacent to an edge of said underlying substrate.

13. The assembly of claim, wherein each hollow microneedle is located adjacent to an edge of said underlying substrate.

14. The assembly of claim 1, wherein said skin interface element and said connector are formed with complementary features deployed such that, when said skin interface element reaches said active position, said complementary features inter-engage to lock said skin interface element in said active position.

15. The assembly of claim 1, wherein said skin interface element and said connector are formed with complementary features inter-engaging to define a path of motion of said skin interface element from said inactive position to said active position, said path of motion including a linear motion of said skin interface element relative to said connector.

16. The assembly of claim 9, wherein said path of motion includes a rotation motion of said skin interface element relative to said connector, and wherein said linear motion is obstructed prior to performance of said rotation motion.

17. The assembly of claim 1, wherein said shield is retained in engagement with said skin interface element so as to move together with said skin interface element as said skin interface element moves from said inactive position to said active position.

18. The assembly of claim 1, wherein said connector is formed with a female Luer taper and an associated resilient locking element configured for permanent attachment to an injection device with a male Luer taper.

19. The assembly of claim 1, wherein said connector is integrally formed with a body of a syringe.

20. The assembly of claim 1, further comprising an injection device having a liquid drug stored in a reservoir interconnected with an outlet port, said outlet port being provided with a projecting hollow needle, said connector being deployed in fixed spatial relation to said projecting hollow needle, wherein said skin interface element includes a septum associated with said flow channel and positioned such that, when said skin interface element assumes said inactive position, a tip of said projecting needle is located within said septum so as to be sealed by said septum, and when said skin interface element assumes said active position, said tip of said projecting needle extends beyond said septum so as to provide said fluid connection with said flow channel.
